(19) United States
(12) Patent Application Publication
(10) Pub. No.: US 2003/0175863 A1
(43) Pub. Date: Sep. 18, 2003

(54) Influenza immunogen and vaccine

(76) Inventor: Ashley J. Birkett, Escondido, CA (US)

Correspondence Address: WELSH & KATZ, LTD, 120 S RIVERSIDE PLAZA, 22ND FLOOR, CHICAGO, IL, 60606, US

(21) Appl. No.: 10/080,299

(22) Filed: Feb. 21, 2002

Related U.S. Application Data
(63) Continuation-in-part of application No. 09/930,915, filed on Aug. 15, 2001.

(51) Int. Cl. C12P 21/06; C12P 19/34; A61K 39/12; A61K 39/29

(52) U.S. Cl. 435/69.1; 424/225.1; 424/227.1; 424/204.1; 435/91.33; 435/91.1

(57) ABSTRACT

A chimeric, carboxy-terminal truncated hepatitis B virus nucleocapsid protein (HBc) is disclosed that contains an immunogen for inducing the production of antibodies to the influenza M2 protein. An immunogenic influenza epitope is preferably expressed at or near the N-terminus or in the HBc immunogenic loop sequence. The chimer preferably contains an influenza-specific T cell epitope and is preferably engineered for both enhanced stability of self-assembled particles and enhanced yield of those chimeric particles. Methods of making and using the chimers are also disclosed.

### DESCRIPTION

### CROSS-REFERENCE TO RELATED APPLICATION

This is a continuation-in-part of application Serial No. 09/930,915, filed Aug. 15, 2001, whose disclosures are incorporated herein by reference.

### TECHNICAL FIELD

The present invention relates to the intersection of the fields of immunology and protein engineering, and particularly to an immunogen and vaccine useful in prevention of influenza infection by influenza A virus.

### BACKGROUND OF THE INVENTION

The family hepadnaviridae are enveloped DNA-containing animal viruses that can cause hepatitis B in humans (HBV). The hepadnavirus family includes hepatitis B viruses of other mammals, e.g., woodchuck (WHV), and ground squirrel (GSHV), and avian viruses found in ducks (DHV) and herons (HeHV). Hepatitis B virus (HBV) used herein refers to a member of the family hepadnaviridae that infects mammals, as compared to a virus that infects an avian host, unless the discussion refers to a specific example of a non-mammalian virus.

The nucleocapsid or core of the mammalian hepatitis B virus (HBV or hepadnavirus) contains a sequence of 183 or 185 amino acid residues, depending on viral subtype, whereas the duck virus capsid contains 262 amino acid residues. Hepatitis B core protein monomers of the several hepadnaviridae self-assemble in infected cells into stable aggregates known as hepatitis B core protein particles (HBc particles). Two three-dimensional structures are reported for C-terminally truncated HBc particles. A first that comprises a minor population contains 90 copies of the HBc subunit protein as dimers or 180 individual monomeric proteins, and a second, major population that contains 120 copies of the HBc subunit protein as dimers or 240 individual monomeric proteins. These particles are referred to as T=4 or T=3 particles, respectively, wherein “T” is the triangulation number. These HBc particles of the human-infecting virus (human virus) are about are about 30 or 34 nm in diameter, respectively. Pumpens et al. (1995) Intervirology, 38:63-74; and Metzger et al. (1998) J. Gen. Viol., 79:587-590.

Conway et al., (1997) Nature, 386:91-94, describe the structure of human HBc particles at 9 \dot{A}ngstrom resolution, as determined from cryo-electron micrographs. Bottcher et al. (1997), Nature, 386:88-91, describe the polypeptide folding for the human HBc monomers, and provide an approximate numbering scheme for the amino acid residues at which alpha-helical regions and their linking loop regions form. Zheng et al., (1992) J. Biol. Chem., 267(13):9422-9429 report that core particle formation is not dependent upon the arginine-rich C-terminal domain, the binding of nucleic acids or the formation of disulfide bonds based on their study of mutant proteins lacking one or more cysteines and others' work with C-terminal-truncated proteins [Birnbaum et al., (1990) J. Virol. 64, 3319-3330]. The low resolution structure of HBc particles reported by Conway et al., (1997) and Bottcher et al., (1997) has been confirmed by a 3.3 Å resolution crystal structure of the T=4 particles reported by Wynne at al., (1999) Mol. Cell, 3(6):70-80.

The hepatitis B nucleocapsid or viral core protein (HBc) has been disclosed as an immunogenic carrier moiety that stimulates the T cell response of an immunized host animal. See, for example, U.S. Pats. No. 4,818,527, U.S. Pat. No. 4,882,145 and U.S. Pat. No. 5,143,726. A particularly useful application of this carrier is its ability to present foreign or heterologous B cell epitopes at the site of the immunodominant loop that is present at about residue positions 70-90, and more usually recited as about positions 75 through 85 from the amino-terminus (N-terminus) of the protein. Clarke et al. (1991) F. Brown et al. eds., Vaccines 91, Cold Spring Harbor Laboratory, Cold Spring Harbor, N.Y., pp.313-318.

During viral replication, HBV nucleocapsids associate with the viral RNA pre-genome, the viral reverse transcriptase (Pol), and the terminal protein (derived from Pol) to form replication competent cores. The association between the nucleocapsid and the viral RNA pre-genome is mediated by an arginine-rich domain at the carboxyl-terminus (C-terminus). When expressed in heterologous expression systems, such as E. coli where viral RNA pre-genome is absent, the protamine-like C-terminus; i.e., residues at positions 150 through 183, can bind E. coli RNA. Zhang et al. (1992) JBC, 267(13) 9422-29.

HBcAg is a particulate protein derived from the hepatitis B virus that has been proposed as a carrier for heterologous epitopes. The relative immunogenicity of HBsAg (HBs) has been compared with HBcAg (HBc), and the ability of each to evoke immune responses in different genetic backgrounds [Milich et al., Science, (1986) 234(4782): p. 1398-1401]. These data emphasize the higher immunogenicity of HBc relative to HBs, and the universal responsiveness to HBc, irrespective of genetic background.

For example, HBc is more than 300 times more immunogenic than HBs in BALB/c mice; and, although both B10.S and B10.M mice are non-responders to HBs, every strain tested is responsive to HBc. These results re-emphasize the suitability of HBc as a vaccine carrier and specifically, its superiority over HBs, hence the selection of HBc as opposed to HBs to carry heterologous epitopes. These facets of HBc are thought to be important in influenza vaccine development, because they address issues of genetic restriction and inadequate antibody titers.

Another advantage of the HBc carrier is the fact that it does not require complex adjuvants for efficacy. This is due to the high inherent immunogenicity of the particle. A comparison of the immunogenicity of HBc-P. berghei particles showed that alum, which is approved for human use, was more effective than either IFA or CFA [Schodel et al., J. Exp. Med., (1994) 180(3): p. 1037-46]. The importance of this observation is highlighted by toxicity problems associated with newer, more complex adjuvants as was recently noted in clinical trials of SKB's candidate malaria vaccine [Stoute et al., N. Engl. J. Med., [1997] 336(2): p. 86-91].

In an application as a vaccine carrier moiety, it may be preferable that the HBV nucleocapsids not bind nucleic acid derived from the host. Birnbaum et al. (1990) J. Virol., 64:3319-3330 showed that the protamine-like C-terminal domain of HBV nucleocapsids could be deleted without interfering with the protein's ability to assemble into virus-like particles. It is thus reported that proteins truncated to about position 144; i.e., containing the HBc sequence from position one through about 144, can self-assemble, whereas deletions beyond residue 139 abrogate capsid assembly [F. Birnbaum & M. Nassal (1990) J. Virl., 64: 3319-30] [Seifer et al., (1995) Intervirology, 38:47-62].

Zlotnick et al., (1997) Proc. Natl. Acad. Sci., USA, 94:9556-9561 studied the assembly of full length and truncated HBc proteins in to particles. In addition to discussing full length molecules, those authors reported the preparation of a truncated protein that contained the HBc sequence from position 1 through 149 in which the cysteines at positions 48, 61 and 107 were each replaced by alanines and in which a cysteine residue was added at the C-terminus (position 150). That C-terminal mercaptan was used for linkage to a gold atom cluster for labeling in electron microscopy.

More recently, Metzger et al. (1998) J. Gen. Viol., 79:587-590 reported that the proline at position 138 (Pro-138 or P138) of the human viral sequence is required for particle formation. Those authors also reported that assembly capability of particles truncated at the carboxy-terminus to lengths of 142 and 140 residues was affected, with assembly capability being completely lost with truncations resulting in lengths of 139 and 137 residues.

Several groups have shown that truncated particles exhibit reduced stability relative to standard hepatitis B core particles [Gallina et al. (1989) J. Virol., 63:4645-4652; Inada, et al. (1989) Virus Res., 14:27-48], evident by variability in particle sizes and the presence of particle fragments in purified preparations [Maassen et al., (1994) Arch. Virol., 135:131-142]. Thus, prior to the report of Metzger et al., above, Pumpens et al., (1995) Intervirology, 38:63-74 summarized the literature reports by stating that the carboxy-terminal border for HBc sequences required for self-assembly was located between amino acid residues 139 and 144, and that the first two or three amino-terminal residues could be replaced by other sequences, but elimination of four or eleven amino-terminal residues resulted in the complete disappearance of chimeric protein in transformed E. coli cells.

Recombinantly-produced hybrid HBc particles bearing internal insertions (referred to in the art as HBc chimeric particles or HBc chimers) containing various inserted polypeptide sequences have been prepared by heterologous expression in a wide variety of organisms, including E. coli, B. subtilis, Vaccinia, Salmonella typhimurium, Saccharomyces cerevisiae. See, for example Pumpens et al. (1995) Intervirology, 38:63-74, and the citations therein that note the work of several research groups. Native HBc particles have also been produced in plants (Tsuda et al., 1998) Vox Sang, 74(3):148-155.

Such HBc chimers often appear to have a less ordered structure, when analyzed by electron microscopy, compared to particles that lack heterologous epitopes [Schodel et al., (1994) J. Exp. Med., 180:1037-1046]. In some cases the insertion of heterologous epitopes into C-terminally truncated HBc particles has such a dramatic destabilizing affect that hybrid particles cannot be recovered following heterologous expression [Schodel et al. (1994) Infect. Immunol., 62:1669-1676]. Thus, many chimeric HBc particles are so unstable that they fall apart during purification to such an extent that they are unrecoverable or they show very poor stability characteristics, making them problematic for vaccine development.

The above Pumpens et al. (1995) Intervirology, 38:63-74 report lists particle-forming chimers in which the inserted polypeptide sequence is at the N-terminus, the C-terminus and between the termini. Insert lengths reported in that article are 24 to 50 residues at the N-terminus, 7 to 43 residues internally, and 11 to 741 residues at the C-terminus.

Kratz et al., (1999) Proc. Natl. Acad. Sci., U.S.A., 96:1915-1920 recently described the E. coli expression of chimeric HBc particles comprised of a truncated HBc sequence internally fused to the 238-residue green fluorescent protein (GFP). This chimer contained the inserted GFP sequence flanked by a pair of glycine-rich flexible linker arms replacing amino acid residues 79 and 80 of HBc. Those particles were said to effectively elicit antibodies against native GFP in rabbits as host animals.

U.S. Pat. No. 5,990,085 describes two fusion proteins formed from an antigenic bovine inhibin peptide fused into (i) the immunogenic loop between residues 78 and 79 and (ii) after residue 144 of carboxy-terminal truncated HBc. Expressed fusion proteins were said to induce the production of anti-inhibin antibodies when administered in a host animal. The titers thirty days after immunization reported in that patent are relatively low, being 1:3000-15,000 for the fusion protein with the loop insertion and 1:100-125 for the insertion after residue 144.

U.S. Pat. No. 6,231,864 teaches the preparation and use of a strategically modified hepatitis B core protein that is linked to a hapten. The modified core protein contains an insert of one to about 40 residues in length that contains a chemically-reactive amino acid residue to which the hapten is pendently linked.

Recently published WO 01/27281 teaches that the immune response to HBc can be changed from a Th1 response to a Th2 response by the presence or absence, respectively, of the C-terminal cysteine-containing sequence of the native molecule. That disclosure also opines that disulfide formation by C-terminal cysteines could help to stabilize the particles. The presence of several residues the native HBc sequence immediately upstream of the C-terminal cysteine was said to be preferred, but not required. One such alternative that might be used to replace a truncated C-terminal HBc sequence was said to include a C-terminal cysteine and an optional sequence that defines an epitope from other than HBc.

Published PCT application WO 01/98333 teaches the deletion of one or more of the four arginine repeats present at the C-terminus of native HBc, while maintaining the C-terminal cysteine residue. That application also teaches that the deleted region can be replaced by an epitope from a protein other than HBc so that the HBc portion of the molecule so formed acts as a carrier for the added epitope.

Published PCT applications corresponding to PCT/US01/25625 and PCT/US01/41759 of the present inventor teach that stabilization of C-terminally truncated HBc particles can be achieved through the use of one or more added cysteine residues in the chimer proteins from which the particles are assembled. Those added cysteine residues are taught to be at or near the C-terminus of the chimeric protein.

A structural feature whereby the stability of full-length HBc particles could be retained, while abrogating the nucleic acid binding ability of full-length HBc particles, would be highly beneficial in vaccine development using the hepadnaviral nucleocapsid delivery system. Indeed, Ulrich et al. in their recent review of the use of HBc chimers as carriers for foreign epitopes [Adv. Virus Res., 50: 141-182 (1998) Academic Press] note three potential problems to be solved for use of those chimers in human vaccines. A first potential problem is the inadvertent transfer of nucleic acids in a chimer vaccine to an immunized host. A second potential problem is interference from preexisting immunity to HBc. A third possible problem relates to the requirement of reproducible preparation of intact chimer particles that can also withstand long-term storage.

The above four published PCT applications appear to contain teachings that can be used to overcome over come the potential problems disclosed by Ulrich et al. As disclosed hereinafter, the present invention provides another HBc chimer that provides unexpectedly high titers of antibodies against influenza, and in one aspect also provides a solution to the problems of HBc chimer stability as well as the substantial absence of nucleic acid binding ability of the construct. In addition, a contemplated recombinant chimer exhibits reducedantigenicity toward preexisting anti-HBc antibodies.

The above particle instability findings related to N-terminal truncated HBc chimer molecules notwithstanding, Neirynck et al., (October 1999) Nature Med., 5(10):1157-1163 reported that particle formation occurred on E. coli expression of a HBc chimer that contained the N-terminal 24-residue portion of the influenza M2 protein fused at residue 5 to full length HBc.

The previously discussed use of hybrid HBc proteins with truncated C-termini for vaccine applications offers several advantages over their full-length counterparts, including enhanced expression levels and lack of bound E. coli RNA. However, C-terminally truncated particles engineered to display heterologous epitopes are often unstable, resulting in particles that either fail to associate into stable particulate structures following expression, or that readily dissociate into non-particulate structures during and/or following purification. Such a lack of stability is exhibited by particles comprised of chimeric HBc molecules that are C-terminally truncated to HBc position 149 and also contain the above residues 1-24 of the influenza A M2 protein.

Others have reported that in wild type hepadnaviral core antigens a cysteine residue upstream of the HBcAg start codon is directly involved in the prevention of particle formation [Schodel et al. (Jan. 15, 1993) J. Biol. Chem., 268(2):1332-1337; Wasenauer et al. (March 1993) J. Virol., 67(3):1315-1322; and Nassal et al. (July 1993) J. Virol., 67(7):4307-4315]. All three groups reported that in wild type HBeAg, the cysteine residue at position −7 of the pre-core sequence, which is present when the core gene is translated from an upstream initiator methionine at position −30, is responsible for preventing particle formation and therefore facilitating the transition from particulate HBcAg to secreted, non-particulate HBeAg.

Based upon the above three publications, one would expect the inclusion of one or more cysteine residues at a position prior to the initiator methionine of HBc; i.e., at a residue position of less than one relative to the N-terminus of the sequence of SEQ ID NO: 1, to actually destabilize hybrid particles rather than stabilize them. As will be seen from the discussion that follows, the present invention provides results that are contrary to those expectations.

### BRIEF SUMMARY OF THE INVENTION

The present invention contemplates an immunogen for inducing antibodies to influenza A, and an inoculum and a vaccine comprising that immunogen dispersed in a physiologically tolerable diluent. A contemplated immunogen is a self-assembled particle comprised of recombinant hepatitis B virus core (HBc) chimer protein molecules. Each of those molecules has a length of about 150 to about 325 amino acid residues and contains four peptide-linked amino acid residue sequence domains from the N-terminus that are denominated Domains I, II, III and IV.

The first domain, Domain I, comprises about 75 to about 110 amino acid residues. The sequence of this Domain includes at least the sequence of the residues of position 4 through position 75 of HBc. One to three cysteine residues are also present at a position in the chimer molecule of about one to about −20 relative to the N-terminus of HBc of SEQ ID NO: 247 [N-terminal cysteine residue(s)]. The one or more N-terminal cysteine residues are present within a sequence other than that of the pre-core sequence of HBc. Domain I can further include a sequence of about 6 to about 24 residues of an influenza A M2 polypeptide X₁X₂X₃X₄X₅X₆X₇X₈TPIRNE X₁₅X₁₆X₁₇X₁₈X₁₉X₂₀-X₂₁X₂₂X₂₃X₂₄ of SEQ ID NO: 9 that are peptide-bonded to or within about 15 residues of the N-terminus of the HBc sequence, and whose subscripted X residues are defined hereinafter, as well as one or more or HBc residues 1-4.

The second domain, Domain II, comprises about 10 to about 60 amino acid residues peptide-bonded to residue 75 of Domain I of which (i) zero to all residues in the sequence of HBc positions 76 through 85 are present peptide-bonded to (ii) an optional sequence of about 6 to about 48 residues that constitute one or more repeats of the above influenza A M2 polypeptide of SEQ ID NO: 9.

The third domain, Domain III, is an HBc sequence from position 86 through position 135 peptide-bonded to residue 85.

The fourth domain, Domain IV, comprises (i) the residues of positions 136-140 plus up to nine residues of an HBc amino acid residue sequence from position 141 through 149 peptide-bonded to the residue of position 135 of Domain III, (ii) zero to three cysteine residues, (iii) fewer than three arginine or lysine residues, or mixtures thereof adjacent to each other, and (iv) up to about 100 amino acid residues in a sequence heterologous to HBc from position 164 to the C-terminus.

A contemplated chimer molecule (i) contains no more than 10 percent conservatively substituted amino acid residues in the HBc sequence, (ii) self-assembles into particles that are substantially free of binding to nucleic acids on expression in a host cell, and those particles are more stable on formation than are particles formed from an otherwise identical HBc chimer that lacks said N-terminal cysteine residue(s) or in which an N-terminal cysteine residue present in the chimer molecule is replaced by another residue.

It is preferred that the HBc sequence of Domain I include the residues of position 4 through position 75 along plus at least an N-terminal cysteine residue. It is further preferred that a contemplated immunogen contain one cysteine residue within Domain IV alone or in an amino acid residue sequence heterologous to that of HBc from position 164 to the C-terminus. It is particularly preferred that that heterologous sequence comprise a T cell epitope of influenza A.

Another embodiment comprises an inoculum or vaccine that comprises an above HBc chimer particle that is dissolved or dispersed in a pharmaceutically acceptable diluent composition that typically also contains water. When administered in an immunogenic effective amount to an animal such as a mammal or bird, an inoculum induces antibodies that immunoreact specifically with the chimer particle. The antibodies so induced also immunoreact specifically with (bind to) the N-terminal portion of the M2 protein.

The present invention has several benefits and advantages.

A particular benefit of the invention is that its use as a vaccine provides extraordinary antibody titers against influenza A.

An advantage of the invention is that those very high antibody titers have been produced with the aid of an adjuvant approved for use in humans.

Another benefit of the invention is that the recombinant immunogen can be prepared easily and in large quantities using well-known cellculturetechniques to grow transformed host cells.

Another advantage of the invention is that the immunogen is easily prepared using well-known recombinant techniques.

Yet another benefit of the invention is that a preferred immunogen exhibits greater stability at elevated temperatures than to other HBc chimers.

Yet another advantage of the invention is that a contemplated immunogen is substantially free of nucleic acids.

Still further benefits and advantages will be apparent to the worker of ordinary skill from the disclosure that follows.

### BRIEF DESCRIPTION OF THE DRAWINGS

In the drawings forming a portion of this disclosure:

FIG. 1, shown in two panels as FIG. 1A and FIG. 1B, provides an alignment of six published sequences for mammalian HBc proteins from six viruses. The first (SEQ ID NO: 1), human viral sequence is of the ayw subtype and was published in Galibert et al. (1983) Nature, 281:646-650; the second human viral sequence (SEQ ID NO: 2), of the adw subtype, was published by Ono et al. (1983) Nucleic Acids Res., 11(6): 1747-1757; the third human viral sequence (SEQ ID NO: 3), is of the adw2 subtype and was published by Valenzuela et al., Animal Virus Genetics, Field et al. eds., Academic Press, New York (1980)pages 57-70; the fourth human viral sequence (SEQ ID NO: 4), is of the adyw subtype that was published by Pasek et al. (1979) Nature, 282:575-579; the fifth sequence (SEQ ID NO: 5), is that of the woodchuck virus that was published by Galibert et al. (1982) J. Virol., 41:51-65; and the sixth mammalian sequence, (SEQ ID NO: 6), is that of the ground squirrel that was published by Seeger et al. (1984) J. Virol.,51:367-375.

FIG. 2 shows the modifications made to commercial plasmid vector pKK223-3 in the preparation of plasmid vector pKK223-3N used herein for preparation of recombinant HBc chimers. The modified sequence (SEQ ID NO: 7) is shown below the sequence of the commercially available vector (SEQ ID NO: 8). The bases of the added NcoI site are shown in lower case letters and the added bases are shown with double underlines, whereas the deleted bases are shown as dashes. The two restriction sites present in this segment of the sequence (NcoI and HindIII) are indicated.

FIG. 3 is an analytical size exclusion chromatography elution profile for ICC-1603 particles in which absorbance at 280 nm is shown on the ordinate and time in seconds is shown on the abscissa.

FIG. 4 is an analytical size exclusion chromatography elution profile for ICC-1590 particles as discussed for FIG. 3.

FIG. 5 is an analytical size exclusion chromatography elution profile for ICC-1560 particles as discussed for FIG. 3.

FIG. 6 is an analytical size exclusion chromatography elution profile for ICC-1605 particles as discussed for FIG. 3.

FIG. 7 is an analytical size exclusion chromatography elution profile for ICC-1604 particles as discussed for FIG. 3.

FIG. 8 is an analytical size exclusion chromatography elution profile for ICC-1438 particles as discussed for FIG. 3.

FIG. 9 is an analytical size exclusion chromatography elution profile for ICC-1492 particles as discussed for FIG. 3.

FIG. 10 is a photograph of an SDS-PAGE analysis under reducing conditions following particle preparation that shows the ICC-1438 monomer construct was unstable after aging (Lane 2) as compared to the ICC-1492 construct (Lane 3), with HBc-149 (Lane 1), ICC-1475 (Lane 4) and ICC-1473 (Lane 5) serving as additional molecular weight controls.

### DEFINITIONS

Numerals utilized in conjunction with HBc chimers indicate the position in the HBc ayw amino acid residue sequence of SEQ ID NO: 1 at which one or more residues has been added to or deleted from the sequence, regardless of whether additions or deletions to the amino acid residue sequence are present. Thus, HBc149 indicates that the chimer ends at residue 149, whereas HBc149+C150 indicates that that same chimer contains a cysteine residue at HBc position 150 relative to the sequence numbers of SEQ ID NO: 1.

The term “antibody” refers to a molecule that is a member of a family of glycosylated proteins called immunoglobulins, which can specifically bind to an antigen.

The word “antigen” has been used historically to designate an entity that is bound by an antibody or receptor, and also to designate the entity that induces the production of the antibody. More current usage limits the meaning of antigen to that entity bound by an antibody or receptor, whereas the word “immunogen” is used for the entity that induces antibody production or binds to the receptor. Where an entity discussed herein is both immunogenic and antigenic, reference to it as either an immunogen or antigen is typically made according to its intended utility.

“Antigenic determinant” refers to the actual structural portion of the antigen that is immunologically bound by an antibody combining site or T-cell receptor. The term is also used interchangeably with “epitope”.

The word “conjugate” as used herein refers to a hapten operatively linked to a carrier protein, as through an amino acid residue side chain.

The term “conservative substitution” as used herein denotes that one amino acid residue has been replaced by another, biologically similar residue. Examples of conservative substitutions include the substitution of one hydrophobic residue such as isoleucine, valine, leucine or methionine for another, or the substitution of one polar residue for another such as between arginine and lysine, between glutamic and aspartic acids or between glutamine and asparagine and the like.

The term “corresponds” in its various grammatical forms as used in relation to peptide sequences means the peptide sequence described plus or minus up to three amino acid residues at either or both of the amino- and carboxy-termini and containing only conservative substitutions in particular amino acid residues along the polypeptide sequence.

The term “Domain” is used herein to mean a portion of a recombinant HBc chimer molecule that is identified by (i) residue position numbering relative to the position numbers of HBcAg subtype ayw as reported by Galibert et al., (1979) Nature, 281:646-650 (SEQ ID NO: 1). The polypeptide portions of at least chimer Domains I, II and III are believed to exist in a similar tertiary form to the corresponding sequences of naturally occurring HBcAg.

As used herein, the term “fusion protein” designates a polypeptide that contains at least two amino acid residue sequences not normally found linked together in nature that are operatively linked together end-to-end (head-to-tail) by a peptide bond between their respective carboxy- and amino-terminal amino acid residues. The fusion proteins of the present invention are HBc chimer molecules that induce the production of antibodies that immunoreact with a polypeptide that corresponds in amino acid residue sequence to the polypeptide portion of the fusion protein.

The phrase “hepatitis B” as used here refers in its broadest context to any member of the family of mammalian hepadnaviridae, as discussed before.

The words “polypeptide” and “peptide” are used interchangeably throughout the specification and designate a linear series of amino acid residues connected one to the other by peptide bonds between the alpha-amino and carboxy groups of adjacent amino acids. Polypeptides can be a variety of lengths, either in their neutral (uncharged) forms or in forms that are salts. It is well understood in the art that amino acid residue sequences contain acidic and basic groups, and that the particular ionization state exhibited by the peptide is dependent on the pH value of the surrounding medium when the peptide is in solution, or that of the medium from which it was obtained if the peptide is in solid form. Thus, “polypeptide” or its equivalent terms is intended to include the appropriate amino acid residue sequence referenced. A peptide or polypeptide is always shown herein from left to right and in the direction from amino-terminus (N-terminus) to carboxy-terminus (C-terminus).

The term “residue” is used interchangeably with the phrase amino acid residue. All amino acid residues identified herein are in the natural or L-configuration. In keeping with standard polypeptide nomenclature, [J. Biol. Chem., 243, 3557-59 (1969)], abbreviations for amino acid residues are as shown in the following Table of Correspondence.

TABLE OF CORRESPONDENCE

| 1-Letter | 3-Letter | AMINO ACID |
|---|---|---|
| Y | Tyr | L-tyrosine |
| G | Gly | glycine |
| F | Phe | L-phenylalanine |
| M | Met | L-methionine |
| A | Ala | L-alanine |
| S | Ser | L-serine |
| I | Ile | L-isoleucine |
| L | Leu | L-leucine |
| T | Thr | L-threonine |
| V | Val | L-valine |
| P | Pro | L-proline |
| K | Lys | L-lysine |
| H | His | L-histidine |
| Q | Gln | L-glutamine |
| E | Glu | L-glutamic acid |
| Z | Glx | L-glutamic acid |
| | | or |
| | | L-glutamine |
| W | Trp | L-tryptophan |
| R | Arg | L-arginine |
| D | Asp | L-aspartic acid |
| N | Asn | L-asparagine |
| B | Asx | L-aspartic acid |
| | | or |
| | | L-asparagine |
| C | Cys | L-cysteine |

### DETAILED DESCRIPTION OF THE INVENTION

The present invention contemplates an immunogen and a vaccine or inoculum comprising that immunogen against the influenza A virus. A contemplated immunogen is a particle comprised of recombinant hepatitis B virus core (HBc) protein chimer molecules with a length of about 150 to about 325 and preferably about 155 to 225 amino acid residues that contains four peptide-linked amino acid residue sequence domains from the N-terminus that are denominated Domains I, II, III and IV. At least one polypeptide containing 6 to about 24 residues of the influenza A M2 polypeptide of SEQ ID NO: 9, as defined hereinbelow, is present peptide-bonded to the chimer molecule.

(a) Domain I comprises about 71 to about 110 amino acid residues whose sequence includes at least the sequence of the residues of position 5 through position 75 of HBc. One to three cysteine residues is(are) also present at a position in the chimer molecule of about one to about −20 relative to the N-terminus of HBc of SEQ ID NO: 247 [N-terminal cysteine residue(s)]. The one or more N-terminal cysteine residues is(are) present within a sequence other than that of the pre-core sequence of HBc.

Domain I can, and preferably does, further include a (i) sequence of 6 to about 24 residues of an above-noted influenza A M2 polypeptide X₁X₂X₃X₄X₅X₆X₇X₈TPIRNEX₁₅X₁₆X₁₇X₁₈X₁₉X₂₀X₂₁X₂₂X₂₃X₂₄ of SEQ ID NO: 9 that are peptide-bonded to or within about 15 residues of the N-terminus of the HBc sequence, as well as (ii) one or more or HBc residues 1-4. In that influenza A M2 polypeptide sequence,

residues X₁ through X₈ are absent or present, and when present are the residues naturally present in the M2 protein sequence that are methionine, serine, leucine, leucine, threonine, glutamic acid, valine, and glutamic acid, respectively, with the proviso that when one subscripted X residue is present, any remaining subscripted X with a higher subscript number up to 8 is also present,

residues X₁₅ and X₁₆ are present or absent, and when present are tryptophan and glycine, respectively,

residues X₁₇ and X₁₉ are present or absent, and when present are independently cysteine, serine, or alanine,

residue X₁₈ is present or absent, and when present is arginine, and

residues X₂₀ through X₂₄ are present or absent, and when present are the residues naturally present in the M2 protein sequence that are asparagine, aspartic acid, serine, serine and aspartic acid respectively, with the proviso that when one subscripted X residue is present, any remaining subscripted X residue with a lower subscript number down to 15 is also present.

(b) Domain II comprises about 10 to about 60 amino acid residues peptide-bonded to residue 75. This sequence includes (i) zero to all of the residues of a sequence of HBc from HBc position 76 through 85 peptide-bonded to (ii) an optional sequence of about 6 to about 48 residues that constitute one or more repeats of the above influenza A M2 polypeptide of SEQ ID NO: 9.

(c) Domain III is an HBc sequence from position 86 through position 135 that is peptide-bonded to residue 85.

d) Domain IV comprises (i) the residues of positions 136-140 plus up to nine residues of an HBc amino acid residue sequence from position 141 through 149 peptide-bonded to the residue of position 135 of Domain III, (ii) zero to three cysteine residues, (iii) fewer than three arginine or lysine residues, or mixtures thereof adjacent to each other, and (iv) up to about 100 amino acid residues in a sequence heterologous to HBc from position 164 to the C-terminus. Thus, Domain IV contains at least the 5 residues of positions 136-140.

A contemplated chimer molecule (i) contains up to about 10 percent conservatively substituted amino acid residues in the HBc sequence, (ii) self-assembles into particles that are substantially free of binding to nucleic acids on expression in a host cell, and those particles are more stable on formation than are particles formed from an otherwise identical HBc chimer that lacks said N-terminal cysteine residue(s) or in which an N-terminal cysteine residue present in the chimer molecule is replaced by another residue.

In examining the length of a contemplated HBc chimer, such a recombinant protein can have a length of about 150 to about 325 amino acid residues. Preferably, that length is about 155 to about 225 residues. More preferably, the length is about 155 to about 170 residues. These differences in length arise from changes in the length of Domains I, II and IV.

HBc chimers having a Domain I that contains more than a deletion of the first three amino-terminal (N-terminal) residues have been reported to result in the complete disappearance of HBc chimer protein in E. coli cells. Pumpens et al., (1995) Intervirology, 38:63-74. On the other hand, a recent study in which an immunogenic 23-mer polypeptide from the influenza M2 protein was fused to the HBc N-terminal sequence reported that the resultant fusion protein formed particles when residues 1-4 of the native HBc sequence were replaced. Neirynck et al. (October 1999) Nature Med., 5(10):1157-1163. Thus, the art teaches that particles can form when an added amino acid sequence is present peptide-bonded the one of residues 1-5 of HBc, whereas particles do not form if no additional sequence is present and more than residues 1-3 are deleted from the N-terminus of HBc.

An N-terminal sequence peptide-bonded to one of the first five N-terminal residues of HBc can contain a sequence of up to about 40 residues that are heterologous to HBc; i.e., a portion of a pre-core sequence can be present in a contemplated chimer molecule. Exemplary sequences include an influenza A B cell or T cell epitope such as are discussed hereinafter, a sequence of another (heterologous) protein such as β-galactosidase as can occur in fusion proteins as a result of the expression system used, or another hepatitis B-related sequence such as that from the Pre-S1 or Pre-S2 regions or the major HbsAg immunogenic sequence.

Domain I preferably has the sequence of residues of positions 2-, 3- or 4- through 75 of HBc. Domain I also contains one to three, preferably one, added cysteine residue(s) and also preferably includes about 6 to about 24 residues of the sequence of the extracellular region of the influenza A M2 protein peptide-bonded at the amino-terminus as discussed herein below. Domain I therefore contains a deletion of at least the methionine residue of position 1 of HBc and can include deletions of the residues at positions 2, 3 and 4.

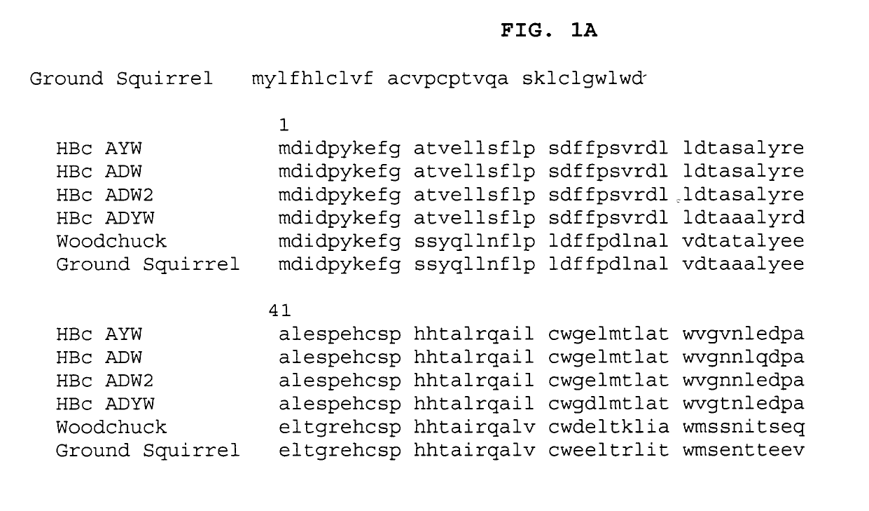

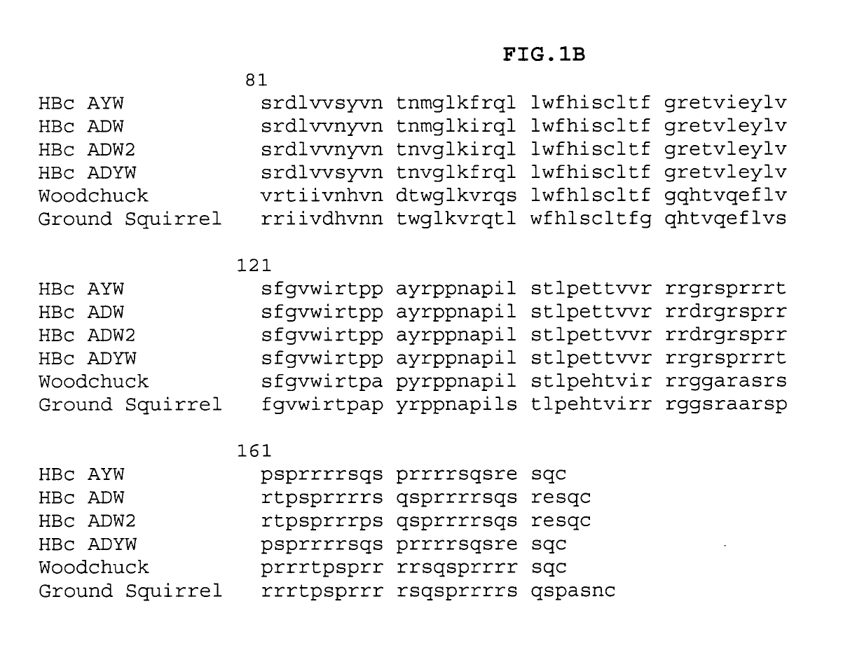

The one or more cysteine residues present in Domain I is(are) located at a position in the chimer molecule of about one to about −20 relative to the N-terminus of HBc of SEQ ID NO: 1 [N-terminal cysteine residue(s)]. Thus, using the sequence of SEQ ID NO: 1 as a reference point, the N-terminal cysteine residue(s) is located in the chimer molecule at a position that corresponds to the methionine at position 1 of SEQ ID NO: 1 (FIG. 1), or at a position up to about 20 residues downstream from that position. More preferably, an N-terminal cysteine is located at a position of about one to about minus 14 relative to position 1 of SEQ ID NO: 1.

The one or more N-terminal cysteine residues are present within a sequence other than that of the pre-core sequence of HBc. As was noted previously, the HBeAg molecule contains the pre-core sequence that includes a cysteine residue. That molecule does not form particles, whereas particles are desired herein. Thus, although an N-terminal cysteine residue can be adjacent to a pre-core sequence, such a residue is not present within a pre-core sequence or a contemplated chimer molecule.

Domain I can have a length of about 110 residues. Preferably, Domain I has a length of about 95 to about 100 amino acid residues, and includes an influenza A M2 polypeptide epitope sequence of SEQ ID NO: 9, that preferably includes the C-terminal 23 residues.

Domain II, which is peptide-bonded to residue 75, contains about 10 to about 60 amino acid residues. This Domain includes zero through all of the sequence of HBc residues of positions 76 through 85. Domain II also optionally includes a sequence of about 6 to about 48 residues that constitute one or more repeats of the before-mentioned influenza A M2 polypeptide of SEQ ID NO: 9. The influenza A M2 polypeptide sequence, when present, is preferably peptide-bonded between HBc residues 78 and 79, and all of the HBc sequence from position 76 through 85 is present.

Preferred influenza A M2 polypeptide sequences for insertion into Domains I or II, or both, of a contemplated recombinant HBc chimer are enumerated in Table A, below. A sequence beginning with a methionine residue (M) is designed to be N-terminal sequence for insertion into the N-terminus of Domain I, whereas a sequence free of an N-terminal M residue is designed for insertion into Domain II.

TABLE A

Influenza A M2 Protein B Cell Epitopes

| | |
|---|---|
| Sequence | SEQ ID NO |

| | |
|---|---|
| SLLTEVETPIRNEWGCRCNGSSD | 10 |
| SLLTEVETPIRNEWGCRCNDSSD | 11 |
| SLLTEVETPIRNEWGARANDSSD | 12 |
| SLLTEVETPIRNEWGSRSNDSSD | 13 |
| SLLTEVETPIRNEWGSRCNDSSD | 14 |
| SLLTEVETPIRNEWGCRSNDSSD | 15 |
| SLLTEVETPIRNEWGCRANDSSD | 16 |
| SLLTEVETPIRNEWGARCNDSSD | 17 |
| MSLLTEVETPIRNEWGCRCNDSSD | 18 |
| MSLLTEVETPIRNEWGSRSNDSSD | 19 |
| MGISLLTEVETPIRNEWGCRCNDSSDELLGWLWGI | 20 |
| MSLLTEVETPIRNEWGARANDSSD | 21 |
| MSLLTEVETPIRNEWGCRANDSSD | 22 |
| MSLLTEVETPIRNEWGARCNDSSD | 23 |
| MSLLTEVETPIRNEWGCRSNDSSD | 24 |
| MSLLTEVETPIRNEWGSRCNDSSD | 25 |
| X₁X₂X₃X₄X₅X₆X₇X₈TPIRNEX₁₅X₁₆X₁₇X₁₈X₁₉X₂₀- | |
| X₂₁X₂₂X₂₃X₂₄ | 9 |
| Influenza B Protein | |
| NNATFNYTNVNPISHIR | 83 |

In the polypeptide of SEQ ID NO: 9, X₁ through X₈ are absent or present, and when present are the residues naturally present in the reported M2 protein sequence; i.e., methionine, serine, leucine, leucine, threonine, glutamic acid, valine, and glutamic acid, respectively, with the proviso that when one subscripted X is present, any remaining subscripted X residue with a higher subscript number up to 8 is also present. Thus, when X₁ is present, each of X₂ through X₈ is also present. Similarly, when X₃ is present, each of X₄ through X₈ is also present, and the like. On the other hand, X₈ can be present without any other of the remaining X residues having a lower valued subscript number being present. The residues X₁₅ and X₁₆ are present or absent, and when present are tryptophan and glycine, respectively. Residues X₁₇ and X₁₉ are present or absent, and when present are independently cysteine, serine, or alanine. It is preferred that one of X₁₇ and X₁₉ be cysteine, particularly when an M2 polypeptide epitope is present at the N-terminus of the chimer molecule. Residue X₁₈ is present or absent, and when present is arginine. Residues X₂₀ through X₂₄ are present or absent, and when present are the residues naturally present in the reported M2 protein sequence; i.e., asparagine, aspartic acid, serine, serine and aspartic acid respectively, with the proviso that when one subscripted X is present, any remaining X residue with a lower subscript number through 15 is also present. Thus, for example, when X₂₃ is present, so are each of residues X₁₅ through X₂₂.

Domain III contains the sequence of HBc position 86 through position 135 peptide-bonded at its N-terminus to residue 85.

The fourth domain, Domain IV, comprises (i) the residues of positions 136 through 140 plus up to nine residues of an HBc amino acid residue sequence from position 141 through 149 peptide-bonded to the residue of position 135 of Domain III, (ii) zero to three cysteine residues, and preferably one cysteine residue, (iii) fewer than three arginine or lysine residues, or mixtures thereof adjacent to each other, and (iv) up to about 100 amino acid residues, preferably up to 50 amino acid residues, and more preferably up to about 25 residues, in a sequence heterologous to HBc from position 164 to the C-terminus.

It is preferred that Domain IV contain up to fourteen residues of an HBc sequence from position 136 through position 149 peptide-bonded to residue 135; i.e., an HBc sequence that begins with the residue of position 136 that can continue through position 149. Thus, if the residue of position 148 is present, so is the sequence of residues of positions 136 through 147, or if residue 141 is present, so is the sequence of residues of positions 136 through 140.

Domain IV can also contain zero to three cysteine residues and those Cys residues are present within about 30 residues of the carboxy-terminus (C-terminus) of the chimer molecule. Preferably, one cysteine (Cys) residue is present, and that Cys is preferably present as the carboxy-terminal (C-terminal) residue, unless an influenza T cell epitope is present as part of Domain IV. When such a T cell epitope is present, the preferred Cys is preferably within the C-terminal last five residues of the HBc chimer.

The presence of the above-discussed N-terminal cysteine residue(s) provides an unexpected enhancement of the ability of the chimer molecules to form stable immunogenic particles (discussed hereinafter). Thus, a contemplated HBc chimer immunogen tends to form particles that stay together upon collection and initial purification as measured by analytical size exclusion chromatography, whose details are discussed hereinafter.

The contemplated particles can also be more stable to decomposition at 37° C. after aging than are similar chimer particles lacking that cysteine residue. This latter type of enhanced stability can be measured using 15% SDS-PAGE gels with particles dispersed in sample buffer (reducing). Gels are stained using Coomassie Blue, and then analyzed. This type of stability is believed to be exhibited against hydrolysis, whereas the stability determined by size exclusion chromatography is that of initial particle formation.

Particles that additionally contain one or more C-terminal cysteine residues exhibit enhanced stability in formation and also toward decomposition on aging, with some particles containing both N- and C-terminal cysteines usually exhibiting greater stability in either measure than those particles having only an added cysteine at either the N- or C-terminus.

Domain IV contains fewer than three arginine or lysine residues, or mixtures thereof adjacent to each other. Arginine and lysines are present in the C-terminal region of HBc that extends from position 164 through the C-terminus of the native molecule. That region is sometimes referred to as the “protamine” or “arginine-rich” region of the molecule and binds nucleic acids. A contemplated HBc chimer molecule and particle are substantially free of bound nucleic acids.

The substantial freedom of nucleic acid binding can be readily determined by a comparison of the absorbance of the particles in aqueous solution measured at both 280 and 260 nm; i.e., a 280/260 absorbance ratio. The contemplated particles do not bind substantially to nucleic acids that are oligomeric and/or polymeric DNA and RNA species originally present in the cells of the organism used to express the protein. Such nucleic acids exhibit an absorbance at 260 nm and relatively less absorbance at 280 nm, whereas a protein such as a contemplated chimer absorbs relatively less at 260 nm and has a greater absorbance at 280 nm.

Thus, recombinantly expressed HBc particles or chimeric HBc particles that contain the arginine-rich sequence at residue positions 150-183 (or 150-185) exhibit a ratio of absorbance at 280 nm to absorbance at 260 nm (280:260 absorbance ratio) of about 0.8, whereas particles free of the arginine-rich nucleic acid binding region of naturally occurring HBc such as those that contain fewer than three arginine or lysine residues or mixtures thereof adjacent to each other, or those having a native or chimeric sequence that ends at about HBc residue position 140 to position 149, exhibit a 280:260 absorbance ratio of about 1.2 to about 1.6.

Chimeric HBc particles of the present invention are substantially free of nucleic acid binding and exhibit a 280:260 absorbance ratio of about 1.2 to about 1.6, and more typically, about 1.4 to about 1.6. This range is due in large part to the number of aromatic amino acid residues present in Domains II and IV of a given chimeric HBc particle. That range is also in part due to the presence of the Cys in Domain IV of a contemplated chimer, whose presence can diminish the observed ratio by about 0.1 for a reason that is presently unknown.

The contemplated chimer HBc particles are more stable in aqueous buffer at 37° C. over a time period of about two weeks to about one month than are particles formed from a HBc chimer containing the same peptide-linked Domain II, III and IV sequences and an otherwise same Domain I sequence in which the one to three cysteine residues [N-terminal cysteine residue(s)] are absent or a single N-terminal residue present is replaced by another residue such as an alanine residue.

Thus, for example, particles containing an influenza A M2 polypeptide in Domain I [e.g. ICC-1590 particles] that include two cysteine residues is more stable than otherwise identical particles [ICC-1603 particles] assembled from chimer molecules whose N-terminal M2 variant sequence contains serine residues in place of the cysteines. Similarly, particles containing the above influenza B cell epitope in Domain I and a single cysteine at the C-terminus [ICC-1605 particles] are more stable than are otherwise identical particles in which that cysteine is absent, but are less stable than are the particles containing the two N-terminal cysteines, ICC-1590 particles or those particles that contained both N-terminal and C-terminal cysteines [ICC-1604 particles].

A contemplated particle containing a N-terminal cysteine residue is also typically prepared in greater yield than is a particle assembled from a chimer molecule lacking a N-terminal cysteine. This increase in yield can be seen from the mass of particles obtained or from integration of traces from analytical gel filtration analysis using Superose® 6 HR as discussed hereinafter.

Although the T cell help afforded by HBc is highly effective in enhancing antibody responses (i.e. B cell-mediated) to ‘carried’ epitopes following vaccination, HBc does not activate influenza-specific T cells, except in restricted individuals for whom the B cell epitope is also a T cell epitope. To help ensure universal priming of influenza-specific T helper cells, in addition to B cells, one or more influenza-specific T helper epitopes is preferably incorporated into a contemplated immunogen and is located in Domain IV of the immunogen.

A plurality of the above or another T cell epitopes can be present in Domain IV or another B cell epitope can be present. In preferred practice, Domain IV has up to about 50 residues in a sequence heterologous to HBc. More preferably, that sequence is up to about 25 residues and includes a T cell epitope.

A contemplated recombinant HBc chimer molecule is typically present and is used in an immunogen or vaccine as a self-assembled particle. These particles are comprised of 180 to 240 chimer molecules that separate into protein molecules in the presence of disulfide reducing agents such as 2-mercaptoethanol, and the individual molecules are therefore thought to be bound together into the particle primarily by disulfide bonds. These particles are similar to the particles observed in patients infected with HBV, but these particles are non-infectious. Upon expression in various prokaryotic and eukaryotic hosts, the individual recombinant HBc chimer molecules assemble in the host into particles that can be readily harvested from the host cells.

In addition to the before-discussed N- and C-truncations and insertion of influenza M2 polypeptide epitopes, a contemplated chimer molecule can also contain conservative substitutions in the amino acid residues that constitute HBc Domains I, II, III and IV. Conservative substitutions are as defined before.

More rarely, a “nonconservative” change, e.g., replacement of a glycine with a tryptophan is contemplated. Analogous minor variations can also include amino acid deletions or insertions, or both. Guidance in determining which amino acid residues can be substituted, inserted, or deleted without abolishing biological activity can be found using computer programs well known in the art, for example LASERGENE software (DNASTAR Inc., Madison, Wis.)

The HBc portion of a chimer molecule of the present invention [the portion having the HBc sequence that has other than a sequence of an added epitope, or heterologous residue(s) that are a restriction enzyme artifact] most preferably has the amino acid residue sequence at positions 2 through 149 of subtype ayw that is shown in FIG. 1 (SEQ ID NO: 1), when present. Somewhat less preferred are the corresponding amino acid residue sequences of subtypes adw, adw2 and adyw that are also shown in FIG. 1 (SEQ ID NOS: 2,3 and 4). Less preferred still are the sequences of woodchuck and ground squirrel at aligned positions 2 through 149 that are the last two sequences of FIG. 1 (SEQ ID NOS: 5 and 6). As noted elsewhere, portions of different sequences from different mammalian HBc proteins can be used together in a single chimer.

When the HBc portion of a chimer molecule of the present invention has other than a sequence of a mammalian HBc molecule at positions 2 through 149, when present, because one or more conservative substitutions has been made, it is preferred that no more than 10 percent, and more preferably no more than 5 percent, and most preferably no more than 3 percent of the amino acid residues are substituted as compared to SEQ ID NO: 1 from position 2 through 149. A contemplated chimer of 149 HBc residues can therefore contain up to about 15 residues that are different from those of SEQ ID NO: 1 at positions 2 through 149, and preferably about 7 or 8 residues. More preferably, up to about 5 residues are different from the ayw sequence (SEQ ID NO: 1) at residue positions 2-149. Where an HBc sequence is truncated further at one or both termini, the number of substituted residues is proportionally different. Deletions elsewhere in the molecule are considered conservative substitutions for purposes of calculation.

Chimer Preparation

A contemplated chimeric immunogen is prepared using the well-known techniques of recombinant DNA technology. Thus, sequences of nucleic acid that encode particular polypeptide sequences are added and deleted from the precursor sequence that encodes HBV.

A contemplated chimeric immunogen typically utilizes a cysteine residue present in the M2 sequence as the N-terminal cysteine. Primers for the preparation of such chimer molecules by in vitro mutagenesis of a polynucleotide encoding an HBc molecule are discussed hereinafter. When a cysteine-containing M2 polypeptide epitope is not present at the N-terminus, the N-terminal cysteine can be provided by in vitro mutagenesis using a primer that encodes just a cysteine-containing portion of the M2 polypeptide or a simple N-terminal start sequence such as Met-Cys- or Met-Gly-Cys-.

As was noted previously, the HBc immunodominant loop is usually recited as being located at about positions 75 through 85 from the amino-terminus (N-terminus) of the intact protein. The influenza A M2 B cell epitope-containing sequence can be placed into that immunodominant loop sequence of Domain II. That placement substantially eliminates the HBc immunogenicity and antigenicity of the HBc loop sequence, while presenting the influenza A M2 B cell epitope in an extremely immunogenic position in the assembled chimer particles.

One of two well-known strategies is particularly useful for placing the influenza A M2 B cell sequence into the loop sequence at a desired location such as between residues 78 and 79. A first, less successful strategy is referred to as replacement in which DNA that codes for a portion of the loop is excised and replaced with DNA that encodes an influenza A M2 B cell sequence. The second strategy is referred to as insertion in which an influenza A M2 B cell sequence is inserted between adjacent residues in the loop.

Site-directed mutagenesis using the polymerase chain reaction (PCR) is used in one exemplary replacement approach to provide a chimeric HBc DNA sequence that encodes a pair of different restriction sites, e.g. EcoRI and SacI, one near each end of the immunodominant loop-encoding DNA. Exemplary residues replaced are 76 through 81. The loop-encoding section is excised, an influenza A M2 B cell epitope-encoding sequence flanked on each side by appropriate HBc sequence residues is ligated into the restriction sites and the resulting DNA is used to express the HBc chimer. See, for example, Table 2 of Pumpens et al., (1995) Intervirology, 38:63-74 for exemplary uses of a similar technique.

Alternatively, a single restriction site or two sites can be encoded into the region, the DNA cut with a restriction enzyme(s) to provide “sticky” or ends, and an appropriate sticky- or blunt-ended heterologous DNA segment ligated into the cut region. Examples of this type of sequence replacement into HBc can be found in the work reported in Schodel et al., (1991) F. Brown et al. eds., Vaccines 91, Cold Spring Harbor Laboratory, Cold Spring Harbor, N.Y., pp.319-325, Schodel et al., Behring Inst. Mitt., 1997(98): p. 114-119 and Schodel et al., J. Exp. Med., (1994) 180(3): p. 1037-4, the latter two papers discussing the preparation of vaccines against malarial pathogens P. yoelii and P. berghei, respectively. A replacement strategy that results in a net removal of residues from the immunodominant loop is usually not used herein.

Insertion is preferred. In an illustrative example of the insertion strategy, site-directed mutagenesis is used to create two restriction sites adjacent to each other and between codons encoding adjacent amino acid residues, such as those at residue positions 78 and 79. This technique adds twelve base pairs that encode four amino acid residues (two for each restriction site) between formerly adjacent residues in the HBc loop.

Upon cleavage with the restriction enzymes, ligation of the DNA coding for the influenza A M2 sequence and expression of the DNA to form HBc chimers, the HBc loop amino acid sequence is seen to be interrupted on its N-terminal side by the two residues encoded by the 5′ restriction site, followed toward the C-terminus by the influenza A M2 B-cell epitope sequence, followed by two more heterologous, non-loop residues encoded by the 3′ restriction site and then the rest of the loop sequence. This same strategy is also preferably used for insertion into Domain IV of a T cell epitope or one or more cysteine residues that are not a part of a T cell epitope. A similar strategy using an insertion between residues 82 and 83 is reported in Schoedel et al., (1990) F. Brown et al. eds., Vaccines 90, Cold Spring Harbor Laboratory, Cold Spring Harbor, N.Y., pp.193-198.

For example, a DNA sequence that encodes a C-terminal truncated HBc sequence (HBc149) is engineered to contain adjacent EcoRI and SacI sites between residues 78 and 79. Cleavage of that DNA with both enzymes provides one fragment that encodes HBc positions 1-78 3′-terminated with an EcoRI sticky end, whereas the other fragment has a 5′-terminal SacI sticky end and encodes residues of positions 79-149. Ligation of a synthetic nucleic acid having a 5′ AATT overhang followed by a sequence that encodes a desired influenza A M2 B cell epitope and a AGCT 3′ overhang provides a HBc chimer sequence that encodes that B cell epitope flanked on each side by two heterologous residues (GI and EL, respectively) between residues 78 and 79, while destroying the EcoRI site and preserving the SacI site.

A similar strategy can be used for insertion of a C-terminal cysteine-containing sequence. Here, EcoRI and HindIII restriction sites are engineered in to the HBc DNA sequence after amino acid residue position 149. After digestion with EcoRI and HindIII, a synthetic DNA having the above AATT 5′ overhang followed by a T cell epitope-encoding sequence, a stop codon and a 3′ AGCT overhang were ligated into the digested sequence to form a sequence that encoded HBc residues 1-149 followed by two heterologous residues (GI), the stop codon and the HindIII site.

PCR amplification using a forward primer having a SacI restriction site followed by a sequence encoding HBc beginning at residue position 79, followed by digestion with SacI and HindIII provided a sequence encoding HBc positions 79-149 plus the two added residues and the T cell epitope at the C-terminus. Digestion of that construct with SacI and ligation provides the complete gene encoding a desired recombinant HBc chimer immunogen having the sequence, from the N-terminus, of HBc positions 1-78, two added residues, the influenza A M2 B cell epitope, two added residues, HBc positions 79-149, two added residues, and the T cell epitope.

It is noted that the preferred use of two heterologous residues on either side of (flanking) a B cell or T cell epitope is a matter of convenience. As a consequence, one can also use zero to three or more added residues that are not part of the HBc sequence on either or both sides of an inserted sequence. One or both ends of the insert and HBc nucleic acid can be “chewed back” with an appropriate nuclease (e.g. S1 nuclease) to provide blunt ends that can be ligated together. Added heterologous residues that are neither part of the inserted B cell or T cell epitopes nor a part of the HBc sequence are not counted in the number of residues present in a recited Domain.

It is also noted that one can also synthesize all or a part of a desired recombinant HBc chimer nucleic acid using well-known synthetic methods as is discussed and illustrated in U.S. Pat. No.5,656,472 for the synthesis of the 177 base pair DNA that encodes the 59 residue ribulose bis-phosphate carboxylase-oxygenase signal peptide of Nicotiana tabacum. For example, one can synthesize Domains I and II with a blunt or “sticky” end that can be ligated to Domains III and IV to provide a construct that expresses a contemplated HBc chimer that contains zero added residues to the N-terminal side of the B cell epitope and zero to three added residues on the C-terminal side or at the Domain II/III junction or at some other desired location.

A nucleic acid sequence (segment) that encodes a previously described HBc chimer molecule or a complement of that coding sequence is also contemplated herein. Such a nucleic acid segment is present in isolated and purified form in some preferred embodiments.

In living organisms, the amino acid residue sequence of a protein or polypeptide is directly related via the genetic code to the deoxyribonucleic acid (DNA) sequence of the gene that codes for the protein. Thus, through the well-known degeneracy of the genetic code additional DNAs and corresponding RNA sequences (nucleic acids) can be prepared as desired that encode the same chimer amino acid residue sequences, but are sufficiently different from a before-discussed gene sequence that the two sequences do not hybridize at high stringency, but do hybridize at moderate stringency.

High stringency conditions can be defined as comprising hybridization at a temperature of about 50°-55° C. in 6×SSC and a final wash at a temperature of 68° C. in 1-3×SSC. Moderate stringency conditions comprise hybridization at a temperature of about 50° C. to about 65° C. in 0.2 to 0.3 M NaCl, followed by washing at about 50° C. to about 55° C. in 0.2×SSC, 0.1% SDS (sodium dodecyl sulfate).

A nucleic sequence (DNA sequence or an RNA sequence) that (1) itself encodes, or its complement encodes, a chimer molecule whose HBc portion from residue position 1 through 136, when present, is that of SEQ ID NOS: 1, 2, 3, 4, 5 or 6 and (2) hybridizes with a DNA sequence of SEQ ID NOS: 77, 78, 79, 80, 81 or 82 at least at moderate stringency (discussed above); and (3) whose HBc sequence shares at least 80 percent, and more preferably at least 90 percent, and even more preferably at least 95 percent, and most preferably 100 percent identity with a DNA sequence of SEQ ID NOS: 77, 78, 79, 80, 81 and 82, is defined as a DNA variant sequence. As is well-known, a nucleic acid sequence such as a contemplated nucleic acid sequence is expressed when operatively linked to an appropriate promoter in an appropriate expression system as discussed elsewhere herein.

An analog or analogous nucleic acid (DNA or RNA) sequence that encodes a contemplated chimer molecule is also contemplated as part of this invention. A chimer analog nucleic acid sequence or its complementary nucleic acid sequence encodes a HBc amino acid residue sequence that is at least 80 percent, and more preferably at least 90 percent, and most preferably is at least 95 percent identical to the HBc sequence portion from residue position 1 through residue position 136 shown in SEQ ID NOS: 1, 2, 3, 4, 5 and 6. This DNA or RNA is referred to herein as an “analog of” or “analogous to” a sequence of a nucleic acid of SEQ ID NOS: 77, 78, 79, 80, 81 and 82, and hybridizes with the nucleic acid sequence of SEQ ID NOS: 77, 78, 79, 80, 81 and 82 or their complements herein under moderate stringency hybridization conditions. A nucleic acid that encodes an analogous sequence, upon suitable transfection and expression, also produces a contemplated chimer.

Different hosts often have preferences for a particular codon to be used for encoding a particular amino acid residue. Such codon preferences are well known and a DNA sequence encoding a desired chimer sequence can be altered, using in vitro mutagenesis for example, so that host-preferred codons are utilized for a particular host in which the enzyme is to be expressed. In addition, one can also use the degeneracy of the genetic code to encode the HBc portion of a sequence of SEQ ID NOS: 1, 2, 3, 4, 5 or 6 that avoids substantial identity with a DNA of SEQ ID NOS: 77, 78, 79, 80, 81 or 82 or their complements. Thus, a useful analogous DNA sequence need not hybridize with the nucleotide sequences of SEQ ID NOS: 77, 78, 79, 80, 81 or 82 or a complement under conditions of moderate stringency, but can still provide a contemplated chimer molecule.

A recombinant nucleic acid molecule such as a DNA molecule, comprising a vector operatively linked to an exogenous nucleic acid segment (e.g., a DNA segment or sequence) that defines a gene that encodes a contemplated chimer, as discussed above, and a promoter suitable for driving the expression of the gene in a compatible host organism, is also contemplated in this invention. More particularly, also contemplated is a recombinant DNA molecule that comprises a vector comprising a promoter for driving the expression of the chimer in host organism cells operatively linked to a DNA segment that defines a gene for the HBc portion of a chimer or a DNA variant that has at least 90 percent identity to the chimer gene of SEQ ID NOS: 77, 78, 79, 80, 81 or 82 and hybridizes with that gene under moderate stringency conditions.

Further contemplated is a recombinant DNA molecule that comprises a vector containing a promoter for driving the expression of a chimer in host organism cells operatively linked to a DNA segment that is an analog nucleic acid sequence that encodes an amino acid residue sequence of a HBc chimer portion that is at least 80 percent identical, more preferably 90 percent identical, and most preferably 95 percent identical to the HBc portion of a sequence of SEQ ID NOS: 1, 2, 3, 4, 5 or 6. That recombinant DNA molecule, upon suitable transfection and expression in a host cell, provides a contemplated chimer molecule.

It is noted that because of the 30 amino acid residue N-terminal sequence of ground squirrel HBc does not align with any of the other HBc sequences, that sequence and its encoding nucleic acid sequences and their complements are not included in the above percentages of identity, nor are the portions of nucleic acid that encode that 30-residue sequence or its complement used in hybridization determinations. Similarly, sequences that are truncated at either or both of the HBc N- and C-termini are not included in identity calculations, nor are those sequences in which residues of the immunodominant loop are removed for insertion of a heterologous epitope. Thus, only those HBc-encoding bases or HBc sequence residues that are present in a chimer molecule are included and compared to an aligned nucleic acid or amino acid residue sequence in the identity percentage calculations.

Inasmuch as the coding sequences for the gene disclosed herein is illustrated in SEQ ID NOS: 77, 78, 79, 80, 81 and 82, isolated nucleic acid segments, preferably DNA sequences, variants and analogs thereof can be prepared by in vitro mutagenesis, as is well known in the art and discussed in Current Protocols In Molecular Biology, Ausabel et al. eds., John Wiley & Sons (New York: 1987) p. 8.1.1-8.1.6, that begin at the initial ATG codon for a gene and end at or just downstream of the stop codon for each gene. Thus, a desired restriction site can be engineered at or upstream of the initiation codon, and at or downstream of the stop codon so that other genes can be prepared, excised and isolated.

As is well known in the art, so long as the required nucleic acid, illustratively DNA sequence, is present, (including start and stop signals), additional base pairs can usually be present at either end of the segment and that segment can still be utilized to express the protein. This, of course, presumes the absence in the segment of an operatively linked DNA sequence that represses expression, expresses a further product that consumes the enzyme desired to be expressed, expresses a product that consumes a wanted reaction product produced by that desired enzyme, or otherwise interferes with expression of the gene of the DNA segment.

Thus, so long as the DNA segment is free of such interfering DNA sequences, a DNA segment of the invention can be about 500 to about 15,000 base pairs in length. The maximum size of a recombinant DNA molecule, particularly an expression vector, is governed mostly by convenience and the vector size that can be accommodated by a host cell, once all of the minimal DNA sequences required for replication and expression, when desired, are present. Minimal vector sizes are well known. Such long DNA segments are not preferred, but can be used.

DNA segments that encode the before-described chimer can be synthesized by chemical techniques, for example, the phosphotriester method of Matteucci et al. (1981) J. Am. Chem. Soc., 103:3185. Of course, by chemically synthesizing the coding sequence, any desired modifications can be made simply by substituting the appropriate bases for those encoding the native amino acid residue sequence. However, DNA segments including sequences discussed previously are preferred.

A contemplated HBc chimer can be produced (expressed) in a number of transformed host systems, typically host cells although expression in acellular, in vitro, systems is also contemplated. These host cellular systems include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g. baculovirus); plant cell systems transformed with virus expression vectors (e.g. cauliflower mosaic virus; tobacco mosaic virus) or with bacterial expression vectors (e.g., Ti plasmid); or appropriately transformed animal cell systems such as CHO or COS cells. The invention is not limited by the host cell employed.

DNA segments containing a gene encoding the HBc chimer are preferably obtained from recombinant DNA molecules (plasmid vectors) containing that gene. Vectors capable of directing the expression of a chimer gene into the protein of a HBc chimer is referred to herein as an “expression vector”.

An expression vector contains expression control elements including the promoter. The chimer-coding gene is operatively linked to the expression vector to permit the promoter sequence to direct RNA polymerase binding and expression of the chimer-encoding gene. Useful in expressing the polypeptide coding gene are promoters that are inducible, viral, synthetic, constitutive as described by Poszkowski et al. (1989) EMBO J., 3:2719 and Odell et al. (1985) Nature, 313:810, as well as temporally regulated, spatially regulated, and spatiotemporally regulated as given in Chua et al. (1989) Science, 244:174-181.

One preferred promoter for use in prokaryotic cells such as E. coli is the Rec 7 promoter that is inducible by exogenously supplied nalidixic acid. A more preferred promoter is present in plasmid vector JHEX25 (available from Promega) that is inducible by exogenously supplied isopropyl-β-D-thiogalacto-pyranoside (IPTG). A still more preferred promoter, the tac promoter, is present in plasmid vector pKK223-3 and is also inducible by exogenously supplied IPTG. The pKK223-3 plasmid can be successfully expressed in a number of E. coli strains, such as XL-1, TB1, BL21 and BLR, using about 25 to about 100 μM IPTG for induction. Surprisingly, concentrations of about 25 to about 50 μM IPTG have been found to provide optimal results in 2 L shaker flasks and fermentors.

Several strains of Salmonella such as S. typhi and S. typhimurium and S. typhimurium-E. coli hybrids have been used to express immunogenic transgenes including prior HBc chimer particles both as sources of the particles for use as immunogens and as live, attenuated whole cell vaccines and inocula, and those expression and vaccination systems can be used herein. See, U.S. Pat. No. 6,024,961; U.S. Pat. No. 5,888,799; U.S. Pat. No. 5,387,744; U.S. Pat. No. 5,297,441; Ulrich et al., (1998) Adv. Virus Res., 50:141-182; Tacket et al., (August 1997) Infect. Immun., 65(8):3381-3385; Schodel et al., (February 1997) Behring Inst. Mitt., 98:114-119; Nardelli-Haefliger et al., (December 1996) Infect. Immun., 64(12):5219-5224; Londono et al., (April 1996) Vaccine, 14(6):545-552, and the citations therein.

Expression vectors compatible with eukaryotic cells, such as those compatible with yeast cells or those compatible with cells of higher plants or mammals, are also contemplated herein. Such expression vectors can also be used to form the recombinant DNA molecules of the present invention. Vectors for use in yeasts such as S. cerivisiae or Pichia pastoris can be episomal or integrating, as is well known. Eukaryotic cell expression vectors are well known in the art and are available from several commercial sources. Normally, such vectors contain one or more convenient restriction sites for insertion of the desired DNA segment and promoter sequences. Optionally, such vectors contain a selectable marker specific for use in eukaryotic cells. Exemplary promoters for use in S. cerevisiae include the S. cerevisiae phosphoglyceric acid kinase (PGK) promoter and the divergent promoters GAL 10 and GAL 1, whereas the alcohol oxidase gene (AOX1) is a useful promoter for Pichia pastoris.

For example, to produce chimers in the methylotrophic yeast, P. pastoris, a gene that encodes a desired chimer is placed under the control of regulatory sequences that direct expression of structural genes in Pichia. The resultant expression-competent forms of those genes are introduced into Pichia cells.

More specifically, the transformation and expression system described by Cregg et al. (1987) Biotechnology, 5:479-485; (1987) Molecular and Cellular Biology, 12:3376-3385 can be used. A gene for a chimer V12.Pf3.1 is placed downstream from the alcohol oxidase gene (AOX1) promoter and upstream from the transcription terminator sequence of the same AOX1 gene. The gene and its flanking regulatory regions are then introduced into a plasmid that carries both the P. pastoris HIS4 gene and a P. pastoris ARS sequence (Autonomously Replicating Sequence), which permit plasmid replication within P. pastoris cells [Cregg et al. (1987) Molecular and Cellular Biology, 12:3376-3385].

The vector also contains appropriate portions of a plasmid such as pBR322 to permit growth of the plasmid in E. coli cells. The resultant plasmid carrying a chimer gene, as well as the various additional elements described above, is illustratively transformed into a his4 mutant of P. pastoris; i.e. cells of a strain lacking a functional histidinol dehydrogenase gene.

After selecting transformant colonies on media lacking histidine, cells are grown on media lacking histidine, but containing methanol as described Cregg et al. (1987) Molecular and Cellular Biology, 12:3376-3385, to induce the AOX1 promoters. The induced AOX1 promoters cause expression of the chimer protein and the production of chimer particles in P. pastoris.

A contemplated chimer gene can also be introduced by integrative transformation, which does not require the use of an ARS sequence, as described by Cregg et al. (1987) Molecular and Cellular Biology, 12:3376-3385.

Production of chimer particles by recombinant DNA expression in mammalian cells is illustratively carried out using a recombinant DNA vector capable of expressing the chimer gene in Chinese hamster ovary (CHO) cells. This is accomplished using procedures that are well known in the art and are described in more detail in Sambrook et al., Molecular Cloning: A Laboratory Manual, 2^{nd} ed., Cold Spring Harbor Laboratories (1989).

In one illustrative example, the simian virus (SV40) based expression vector, pKSV-10 (Pharmacia Fine Chemicals, Piscataway, N.J.), is subjected to restriction endonuclease digestion by NcoI and HindIII. A NcoI/HindIII sequence fragment that encodes the desired HBc chimer prepared as described in Example 1 is ligated into the expression plasmid, which results in the formation of a circular recombinant expression plasmid denominated pSV-Pf.

The expression plasmid pSV-Pf contains an intact E. coli ampicillin resistance gene. E. coli RR101 (Bethesda Research Laboratories, Gaithersburg, Md.), when transformed with pSV-Pf, can thus be selected on the basis of ampicillin resistance for those bacteria containing the plasmid. Plasmid-containing bacteria are then cloned and the clones are subsequently screened for the proper orientation of the inserted coding gene into the expression vector.

The above obtained plasmid, pSV-Pf, containing the gene that encodes a desired HBc chimer is propagated by culturing E. coli containing the plasmid. The plasmid DNA is isolated from E. coli cultures as described in Sambrook et al., above.

Expression of a chimer is accomplished by the introduction of pSV-Pf into the mammalian cell line, e.g., CHO cells, using the calcium phosphate-mediated transfection method of Graham et al.(1973) Virol., 52:456, or a similar technique.

To help ensure maximal efficiency in the introduction of pSV-Pf into CHO cells in culture, the transfection is carried out in the presence of a second plasmid, pSV2NEO (ATCC #37149) and the cytotoxic drug G418 (GIBCO Laboratories, Grand Island, N.Y.) as described by Southern et al. (1982) J. Mol. Appl. Genet., 1:327. Those CHO cells that are resistant to G418 are cultured, have acquired both plasmids, pSV2NEO and pSV-Pf, and are designated CHO/pSV-Pf cells. By virtue of the genetic architecture of the pSV-Pf expression vector, a chimer is expressed in the resulting CHO/pSV-Pf cells and can be detected in and purified from the cytoplasm of these cells. The resulting composition containing cellular protein is separated on a column as discussed elsewhere herein.

The choice of which expression vector and ultimately to which promoter a chimer-encoding gene is operatively linked depends directly on the functional properties desired, e.g. the location and timing of protein expression, and the host cell to be transformed. These are well known limitations inherent in the art of constructing recombinant DNA molecules. However, a vector useful in practicing the present invention can direct the replication, and preferably also the expression (for an expression vector) of the chimer gene included in the DNA segment to which it is operatively linked.

In one preferred embodiment, the host that expresses the chimer is the prokaryote, E. coli, and a preferred vector includes a prokaryotic replicon; i.e., a DNA sequence having the ability to direct autonomous replication and maintenance of the recombinant DNA molecule extrachromosomally in a prokaryotic host cell transformed therewith. Such replicons are well known in the art.

Those vectors that include a prokaryotic replicon can also include a prokaryotic promoter region capable of directing the expression of a contemplated HBc chimer gene in a host cell, such as E. coli, transformed therewith. Promoter sequences compatible with bacterial hosts are typically provided in plasmid vectors containing one or more convenient restriction sites for insertion of a contemplated DNA segment. Typical of such vector plasmids are pUC8, pUC9, and pBR329 available from Biorad Laboratories, (Richmond, Calif.) and pPL and pKK223-3 available from Pharmacia, Piscataway, N.J.

Typical vectors useful for expression of genes in cells from higher plants and mammals are well known in the art and include plant vectors derived from the tumor-inducing (Ti) plasmid of Agrobacterium tumefaciens described by Rogers et al. (1987) Meth. in Enzymol., 153:253-277 and mammalian expression vectors pKSV-10, above, and pCI-neo (Promega Corp., #E1841, Madison, Wis.). However, several other expression vector systems are known to function in plants including pCaMVCN transfer control vector described by Fromm et al. (1985) Proc. Natl. Acad. Sci. USA, 82:58-24. Plasmid pCaMVCN (available from Pharmacia, Piscataway, N.J.) includes the cauliflower mosaic virus CaMV 35S promoter.

The above plant expression systems typically provide systemic or constitutive expression of an inserted transgene. Systemic expression can be useful where most or all of a plant is used as the source to a contemplated chimer molecule or resultant particles or where a large part of the plant is used to provide an oral vaccine. However, it can be more efficacious to express a chimer molecule or particles in a plant storage organ such as a root, seed or fruit from which the particles can be more readily isolated or ingested.

One manner of achieving storage organ expression is to use a promoter that expresses its controlled gene in one or more preselected or predetermined non-photosynthetic plant organs. Expression in one or more preselected storage organs with little or no expression in other organs such as roots, seed or fruit versus leaves or stems is referred to herein as enhanced or preferential expression. An exemplary promoter that directs expression in one or more preselected organs as compared to another organ at a ratio of at least 5:1 is defined herein as an organ-enhanced promoter. Expression in substantially only one storage organ and substantially no expression in other storage organs is referred to as organ-specific expression; i.e., a ratio of expression products in a storage organ relative to another of about 100:1 or greater indicates organ specificity. Storage organ-specific promoters are thus members of the class of storage organ-enhanced promoters.

Exemplary plant storage organs include the roots of carrots, taro or manioc, potato tubers, and the meat of fruit such as red guava, passion fruit, mango, papaya, tomato, avocado, cherry, tangerine, mandarin, palm, melons such cantaloupe and watermelons and other fleshy fruits such as squash, cucumbers, mangos, apricots, peaches, as well as the seeds of maize (corn), soybeans, rice, oil seed rape and the like.

The CaMV 35S promoter is normally deemed to be a constitutive promoter. However, recent research has shown that a 21-bp region of the CaMV 35S promoter, when operatively linked into another, heterologous usual green tissue promoter, the rbcS-3A promoter, can cause the resulting chimeric promoter to become a root-enhanced promoter. That 21-bp sequence is disclosed in U.S. Pat. No. 5,023,179. The chimeric rbcS-3A promoter containing the 21-bp insert of U.S. Pat. No. 5,023,179 is a useful root-enhanced promoter herein.

A similar root-enhanced promoter, that includes the above 21-bp segment is the −90 to +8 region of the CAMV 35S promoter itself. U.S. Pat. No. 5,110,732 discloses that that truncated CaMV 35S promoter provides enhanced expression in roots and the radical of seed, a tissue destined to become a root. That promoter is also useful herein.

Another useful root-enhanced promoter is the −1616 to −1 promoter of the oil seed rape (Brassica napus L.) gene disclosed in PCT/GB92/00416 (WO 91/13922 published Sep. 19, 1991). E. coli DH5.alpha. harboring plasmid pRlambdaS4 and bacteriophage lambda.beta.l that contain this promoter were deposited at the National Collection of Industrial and Marine Bacteria, Aberdeen, GB on Mar. 8, 1990 and have accession numbers NCIMB40265 and NCIMB40266. A useful portion of this promoter can be obtained as a 1.0 kb fragment by cleavage of the plasmid with HaeIII.

A preferred root-enhanced promoter is the mannopine synthase (mas) promoter present in plasmid pKan2 described by DiRita and Gelvin (1987) Mol. Gen. Genet, 207:233-241. This promoter is removable from its plasmid pKan2 as a XbaI-XbalI fragment.

The preferred mannopine synthase root-enhanced promoter is comprised of the core mannopine synthase (mas) promoter region up to position −138 and the mannopine synthase activator from −318 to −213, and is collectively referred to as AmasPmas. This promoter has been found to increase production in tobacco roots about 10- to about 100-fold compared to leaf expression levels.

Another root specific promoter is the about 500 bp 5′ flanking sequence accompanying the hydroxyproline-rich glycopeprotein gene, HRGPnt3, expressed during lateral root initiation and reported by Keller et al. (1989) Genes Dev., 3:1639-1646. Another preferred root-specific promoter is present in the about −636 to −1 5′ flanking region of the tobacco root-specific gene ToRBF reported by Yamamoto et al. (1991) Plant Cell, 3:371-381. The cis-acting elements regulating expression are more specifically located by those authors in the region from about −636 to about −299 5′ from the transcription initiation site. Yamamoto et al. reported steady state mRNA production from the ToRBF gene in roots, but not in leaves, shoot meristems or stems.

Still another useful storage organ-specific promoter are the 5′ and 3′ flanking regions of the fruit-ripening gene E8 of the tomato, Lycopersicon esculentum. These regions and their cDNA sequences are illustrated and discussed in Deikman et al. (1988) EMBO J., 7(11):3315-3320 and (1992) Plant Physiol., 100:2013-2017.

Three regions are located in the 2181 bp of the 5′ flanking sequence of the gene and a 522 bp sequence 3′ to the poly (A) addition site appeared to control expression of the E8 gene. One region from −2181 to −1088 is required for activation of E8 gene transcription in unripe fruit by ethylene and also contributes to transcription during ripening. Two further regions, −1088 to −863 and −409 to −263, are unable to confer ethylene responsiveness in unripe fruit but are sufficient for E8 gene expression during ripening.

The maize sucrose synthase-1 (Sh) promoter that in corn expresses its controlled enzyme at high levels in endosperm, at much reduced levels in roots and not in green tissues or pollen has been reported to express a chimeric reporter gene, β-glucuronidase (GUS), specifically in tobacco phloem cells that are abundant in stems and roots. Yang et al. (1990) Proc. Natl. Acad. Sci., U.S.A., 87:4144-4148. This promoter is thus useful for plant organs such as fleshy fruits like melons, e.g. cantaloupe, or seeds that contain endosperm and for roots that have high levels of phloem cells.

Another exemplary tissue-specific promoter is the lectin promoter, which is specific for seed tissue. The lectin protein in soybean seeds is encoded by a single gene (Le1) that is only expressed during seed maturation and accounts for about 2 to about 5 percent of total seed mRNA. The lectin gene and seed-specific promoter have been fully characterized and used to direct seed specific expression in transgenic tobacco plants. See, e.g., Vodkin et al. (1983) Cell, 34:1023 and Lindstrom et al. (1990) Developmental Genetics, 11:160.

A particularly preferred tuber-specific expression promoter is the 5′ flanking region of the potato patatin gene. Use of this promoter is described in Twell et al. (1987) Plant Mol. Biol., 9:365-375. This promoter is present in an about 406 bp fragment of bacteriophage LPOTI. The LPOTI promoter has regions of over 90 percent homology with four other patatin promoters and about 95 percent homology over all 400 bases with patatin promoter PGT5. Each of these promoters is useful herein. See, also, Wenzler et al. (1989) Plant Mol. Biol., 12:41-50.

Still further organ-enhanced and organ-specific promoter are disclosed in Benfey et al. (1988) Science, 244:174-181.

Each of the promoter sequences utilized is substantially unaffected by the amount of chimer molecule or particles in the cell. As used herein, the term “substantially unaffected” means that the promoter is not responsive to direct feedback control (inhibition) by the chimer molecules or particles accumulated in transformed cells or transgenic plant.

Transfection of plant cells using Agrobacterium tumefaciens is typically best carried out on dicotyledonous plants. Monocots are usually most readily transformed by so-called direct gene transfer of protoplasts. Direct gene transfer is usually carried out by electroportation, by polyethyleneglycol-mediated transfer or bombardment of cells by microprojectiles carrying the needed DNA. These methods of transfection are well-known in the art and need not be further discussed herein. Methods of regenerating whole plants from transfected cells and protoplasts are also well-known, as are techniques for obtaining a desired protein from plant tissues. See, also, U.S. Pat. No. 5,618,988 and 5,679,880 and the citations therein.

A transgenic plant formed using Agrobacterium transformation, electroportation or other methods typically contains a single gene on one chromosome. Such transgenic plants can be referred to as being heterozygous for the added gene. However, inasmuch as use of the word “heterozygous” usually implies the presence of a complementary gene at the same locus of the second chromosome of a pair of chromosomes, and there is no such gene in a plant containing one added gene as here, it is believed that a more accurate name for such a plant is an independent segregant, because the added, exogenous chimer molecule-encoding gene segregates independently during mitosis and meiosis. A transgenic plant containing an organ-enhanced promoter driving a single structural gene that encodes a contemplated HBc chimeric molecule; i.e., an independent segregant, is a preferred transgenic plant.

More preferred is a transgenic plant that is homozygous for the added structural gene; i.e., a transgenic plant that contains two added genes, one gene at the same locus on each chromosome of a chromosome pair. A homozygous transgenic plant can be obtained by sexually mating (selfing) an independent segregant transgenic plant that contains a single added gene, germinating some of the seed produced and analyzing the resulting plants produced for enhanced chimer particle accumulation relative to a control (native, non-transgenic) or an independent segregant transgenic plant. A homozygous transgenic plant exhibits enhanced chimer particle accumulation as compared to both a native, non-transgenic plant and an independent segregant transgenic plant.

It is to be understood that two different transgenic plants can also be mated to produce offspring that contain two independently segregating added, exogenous (heterologous) genes. Selfing of appropriate progeny can produce plants that are homozygous for both added, exogenous genes that encode a chimeric HBc molecule. Back-crossing to a parental plant and out-crossing with a non-transgenic plant are also contemplated.

A transgenic plant of this invention thus has a heterologous structural gene that encodes a contemplated chimeric HBc molecule. A preferred transgenic plant is an independent segregant for the added heterologous chimeric HBc structural gene and can transmit that gene to its progeny. A more preferred transgenic plant is homozygous for the heterologous gene, and transmits that gene to all of its offspring on sexual mating.

Inasmuch as a gene that encodes a chimeric HBc molecule does not occur naturally in plants, a contemplated transgenic plant accumulates chimeric HBc molecule particles in a greater amount than does a non-transformed plant of the same type or strain when both plants are grown under the same conditions.

The phrase “same type” or “same strain” is used herein to mean a plant of the same cross as or a clone of the untransformed plant. Where alleic variations among siblings of a cross are small, as with extensively inbred plant, comparisons between siblings can be used or an average arrived at using several siblings. Otherwise, clones are preferred for the comparison.

Seed from a transgenic plant is grown in the field greenhouse, window sill or the like, and resulting sexually mature transgenic plants are self-pollinated to generate true breeding plants. The progeny from these plants become true breeding lines that are evaluated for chimeric HBc molecule particle accumulation, preferably in the field, under a range of environmental conditions.

A transgenic plant homozygous for chimeric HBc molecule particle accumulation is crossed with a parent plant having other desired traits. The progeny, which are heterozygous or independently segregatable for chimeric HBc molecule particle accumulation, are backcrossed with one or the other parent to obtain transgenic plants that exhibit chimeric HBc molecule particle accumulation and the other desired traits. The backcrossing of progeny with the parent may have to be repeated more than once to obtain a transgenic plant that possesses a number of desirable traits.

An insect cell system can also be used to express a HBc chimer. For example, in one such system Autographa californica nuclear polyhedrosis virus (AcNPV) or baculovirus is used as a vector to express foreign genes in Spodoptera frugiperda cells or in Trichoplusia larvae.

The sequences encoding a chimer can be cloned into a non-essential region of the virus, such as the polyhedrin gene, and placed under control of the polyhedrin promoter. Successful insertion of chimer sequence renders the polyhedrin gene inactive and produces recombinant virus lacking coat protein. The recombinant viruses can then be used to infect, for example, S. Frugiperda cells or Trichoplusia larvae in which the HBc chimer can be expressed. E. Engelhard et al. (1994) Proc. Natl. Acad. Sci., USA, 91:3224-3227; and V. Luckow, Insect Cell Expression Technology, pp. 183-218, in Protein Engineering: Principles and Practice, J. L. Cleland et al. eds., Wiley-Liss, Inc, 1996). Heterologous genes placed under the control of the polyhedrin promoter of the Autographa californica nuclear polyhedrosis virus (AcNPV) are often expressed at high levels during the late stages of infection.

Recombinant baculoviruses containing the chimeric gene are constructed using the baculovirus shuttle vector system (Luckow et al. (1993) J. Virol., 67:4566-4579], sold commercially as the Bac-To-Bac™ baculovirus expression system (Life Technologies). Stocks of recombinant viruses are prepared and expression of the recombinant protein is monitored by standard protocols (O'Reilly et al., Baculovirus Expression Vectors: A Laboratory Manual, W. H. Freeman and Company, New York, 1992; and King et al., The Baculovirus Expression System: A Laboratory Guide, Chapman & Hall, London, 1992).

A variety of methods have been developed to operatively link DNA to vectors via complementary cohesive termini or blunt ends. For instance, complementary homopolymer tracts can be added to the DNA segment to be inserted into the vector DNA. The vector and DNA segment are then joined by hydrogen bonding between the complementary homopolymeric tails to form recombinant DNA molecules.

Alternatively, synthetic linkers containing one or more restriction endonuclease sites can be used to join the DNA segment to the expression vector, as noted before. The synthetic linkers are attached to blunt-ended DNA segments by incubating the blunt-ended DNA segments with a large excess of synthetic linker molecules in the presence of an enzyme that is able to catalyze the ligation of blunt-ended DNA molecules, such as bacteriophage T4 DNA ligase.

Thus, the products of the reaction are DNA segments carrying synthetic linker sequences at their ends. These DNA segments are then cleaved with the appropriate restriction endonuclease and ligated into an expression vector that has been cleaved with an enzyme that produces termini compatible with those of the synthetic linker. Synthetic linkers containing a variety of restriction endonuclease sites are commercially available from a number of sources including New England BioLabs, Beverly, Mass. A desired DNA segment can also be obtained using PCR technology in which the forward and reverse primers contain desired restriction sites that can be cut after amplification so that the gene can be inserted into the vector. Alternatively PCR products can be directly cloned into vectors containing T-overhangs (Promega Corp., A3600, Madison, Wis.) as is well known in the art.

The expressed chimeric protein self-assembles into particles within the host cells, whether in single cells or in cells within a multicelled host. The particle-containing cells are harvested using standard procedures, and the cells are lysed using a French pressure cell, lysozyme, sonicator, bead beater or a microfluidizer (Microfluidics International Corp., Newton Mass.). After clarification of the lysate, particles are precipitated with 45% ammonium sulfate, resuspended in 20 mM sodium phosphate, pH 6.8 and dialyzed against the same buffer. The dialyzed material is clarified by brief centrifugation and the supernatant subjected to gel filtration chromatography using Sepharose® CL-4B. Particle-containing fractions are identified, subjected to hydroxyapatite chromatography, and reprecipitated with ammonium sulfate prior to resuspension, dialysis and sterile filtration and storage at −70° C.

Inocula and Vaccines

A before-described recombinant HBc chimer immunogen preferably in particulate form is dissolved or dispersed in an immunogenic effective amount in a pharmaceutically acceptable vehicle composition that is preferably aqueous to form an inoculum or vaccine. When administered to a host animal in need of immunization or in which antibodies are desired to be induced such as a mammal (e.g., a mouse, dog, goat, sheep, horse, bovine, monkey, ape, or human) or bird (e.g., a chicken, turkey, duck or goose), an inoculum induces antibodies that immunoreact with the influenza A M2 B cell epitope present in the immunogen. In a vaccine, those induced antibodies also believed to immunoreact in vivo with (bind to) the virus or virally-infected cells and protect the host from influenza infection. A composition that is a vaccine in one animal can be an inoculum an inoculum for another host, as where the antibodies are induced in a second host that is not infected by influenza A.

The amount of recombinant HBc chimer immunogen utilized in each immunization is referred to as an immunogenic effective amount and can vary widely, depending inter alia, upon the recombinant HBc chimer immunogen, animal host immunized, and the presence of an adjuvant in the vaccine, as discussed below. Immunogenic effective amounts for a vaccine and an inoculum provide the protection or antibody activity, respectively, discussed hereinbefore.

Vaccines or inocula typically contain a recombinant HBc chimer immunogen concentration of about 1 microgram to about 1 milligram per inoculation (unit dose), and preferably about 10 micrograms to about 50 micrograms per unit dose. Immunizations in mice typically contain 10 or 20 μg of chimer particles.

The term “unit dose” as it pertains to a vaccine or inoculum of the present invention refers to a physically discrete unit suitable as an unitary dosage for animals, each unit containing a predetermined quantity of active material calculated to individually or collectively produce the desired immunogenic effect in association with the required diluent; i.e., carrier, or vehicle. A single unit dose or a plurality of unit doses can be used to provide an immunogenic effective amount of recombinant HBc chimer immunogen particles.

Vaccines or inocula are typically prepared from a recovered recombinant HBc chimer immunogen particles by dispersing the particles in a physiologically tolerable (acceptable) diluent vehicle such as water, saline phosphate-buffered saline (PBS), acetate-buffered saline (ABS), Ringer's solution or the like to form an aqueous composition. The diluent vehicle can also include oleaginous materials such as peanut oil, squalane or squalene as is discussed hereinafter.

The immunogenic active ingredient is often mixed with excipients that are pharmaceutically acceptable and compatible with the active ingredient. Suitable excipients are, for example, water, saline, dextrose, glycerol, ethanol, or the like and combinations thereof. In addition, if desired, an inoculum or vaccine can contain minor amounts of auxiliary substances such as wetting or emulsifying agents, pH buffering agents that enhance the immunogenic effectiveness of the composition.

A contemplated vaccine or inoculum advantageously also includes an adjuvant. Suitable adjuvants for vaccines and inocula of the present invention comprise those adjuvants that are capable of enhancing the antibody responses against B cell epitopes of the chimer, as well as adjuvants capable of enhancing cell mediated responses towards T cell epitopes contained in the chimer, if present. Adjuvants are well known in the art (see, for example, Vaccine Design—The Subunit and Adjuvant Approach, 1995, Pharmaceutical Biotechnology, Volume 6, Eds. Powell, M. F., and Newman, M. J., Plenum Press, New York and London, ISBN 0-306-44867-X).

Exemplary adjuvants include complete Freund's adjuvant (CFA) that is not used in humans, incomplete Freund's adjuvant (IFA), squalene, squalane and alum [e.g., Alhydrogel™ (Superfos, Denmark)], which are materials well known in the art, and are available commercially from several sources.

Preferred adjuvants for use with immunogens of the present invention include aluminum or calcium salts (for example hydroxide or phosphate salts). A particularly preferred adjuvant for use herein is an aluminum hydroxide gel such as Alhydrogel™. For aluminum hydroxide gels (alum), the chimer protein is admixed with the adjuvant so that about 50 to about 800 micrograms of aluminum are present per dose, and preferably about 400 to about 600 micrograms are present.

Another particularly preferred adjuvant for use with an immunogen of the present invention is an emulsion. A contemplated emulsion can be an oil-in-water emulsion or a water-in-oil emulsion. In addition to the immunogenic chimer protein particles, such emulsions comprise an oil phase of squalene, squalane, peanut oil or the like as are well-known, and a dispersing agent. Non-ionic dispersing agents are preferred and such materials include mono- and di-C₁₂-C₂₄-fatty acid esters of sorbitan and mannide such as sorbitan mono-stearate, sorbitan mono-oleate and mannide mono-oleate. An immunogen-containing emulsion is administered as an emulsion.

Preferably, such emulsions are water-in-oil emulsions that comprise squalene and mannide mono-oleate (Arlacel™ A), optionally with squalane, emulsified with the chimer protein particles in an aqueous phase. Well-known examples of such emulsions include Montanide™ ISA-720, and Montanide™ ISA 703 (Seppic, Castres, France), each of which is understood to contain both squalene and squalane, with squalene predominating in each, but to a lesser extent in Montanide™ ISA 703. Most preferably, Montanide™ ISA-720 is used, and a ratio of oil-to-water of 7:3 (w/w) is used. Other preferred oil-in-water emulsion adjuvants include those disclosed in WO 95/17210 and EP 0 399 843.

The use of small molecule adjuvants is also contemplated herein. One type of small molecule adjuvant useful herein is a 7-substituted-8-oxo- or 8-sulfo-guanosine derivative described in U.S. Pats. No. 4,539,205, U.S. Pat. No. 4,643,992, U.S. Pat. No. 5,011,828 and U.S. Pat. No. 5,093,318, whose disclosures are incorporated by reference. Of these materials, 7-allyl-8-oxoguanosine (loxoribine) is particularly preferred. That molecule has been shown to be particularly effective in inducing an antigen- (immunogen-) specific response.

Still further useful adjuvants include monophosphoryl lipid A (MPL) available from Corixa Corp. (see, U.S. Pat. No. 4,987,237), CPG available from Coley Pharmaceutical Group, QS21 available from Aquila Biopharmaceuticals, Inc., SBAS2 available from SKB, the so-called muramyl dipeptide analogues described in U.S. Pat. No. 4,767,842, and MF59 available from Chiron Corp. (see, U.S. Pats. No. 5,709,879 and U.S. Pat. No. 6,086,901).

More particularly, immunologically active saponin fractions having adjuvant activity derived from the bark of the South American tree Quillaja Saponaria Molina (e.g. Quil™ A) are also useful. Derivatives of Quil™ A, for example QS21 (an HPLC purified fraction derivative of Quil™ A), and the method of its production is disclosed in U.S. Pat. No. 5,057,540. In addition to QS21 (known as QA21), other fractions such as QA17 are also disclosed.

3-De-O-acylated monophosphoryl lipid A is a well-known adjuvant manufactured by Ribi Immunochem, Hamilton, Mont. The adjuvant contains three components extracted from bacteria: monophosphoryl lipid (MPL) A, trehalose dimycolate (TDM) and cell wall skeleton (CWS) (MPL+TDM+CWS) in a 2% squalene/Tween® 80 emulsion. This adjuvant can be prepared by the methods taught in GB 2122204B. A preferred form of 3-de-O-acylated monophosphoryl lipid A is in the form of an emulsion having a small particle size less than 0.2 μm in diameter (EP 0 689 454 B1).

The muramyl dipeptide adjuvants include N-acetyl-muramyl-L-threonyl-D-isoglutamine(thur-MDP), N-acetyl-nor-muramyl-L-alanyl-D-isoglutamine [CGP 11637, referred to as nor-MDP], and N-acetylmuramyl-L-alanyl-D-isoglutaminyl-L-alanine-2-(1′-2′-dipalmityol-sn-glycero-3-hydroxyphosphoryloxy)-ethylamin [(CGP) 1983A, referred to as MTP-PE].

Preferred adjuvant mixtures include combinations of 3D-MPL and QS21 (EP 0 671 948 B1), oil-in-water emulsions comprising 3D-MPL and QS21 (WO 95/17210, PCT/EP98/05714), 3D-MPL formulated with other carriers (EP 0 689 454 B1), QS21 formulated in cholesterol-containing liposomes (WO 96/33739), or immunostimulatory oligonucleotides (WO 96/02555). Alternative adjuvants include those described in WO 99/52549 and non-particulate suspensions of polyoxyethylene ether (UK Patent Application No. 9807805.8).

Adjuvants are utilized in an adjuvant amount, which can vary with the adjuvant, host animal and recombinant HBc chimer immunogen. Typical amounts can vary from about 1 μg to about 1 mg per immunization. Those skilled in the art know that appropriate concentrations or amounts can be readily determined.

Inocula and vaccines are conventionally administered parenterally, by injection, for example, either subcutaneously or intramuscularly. Additional formulations that are suitable for other modes of administration include suppositories and, in some cases, oral formulation or by nasal spray. For suppositories, traditional binders and carriers can include, for example, polyalkalene glycols or triglycerides; such suppositories may be formed from mixtures containing the active ingredient in the range of 0.5% to 10%, preferably 1-2%. Oral formulations include such normally employed excipients as, for example, pharmaceutical grades of mannitol, lactose, starch, magnesium stearate, sodium saccharine, cellulose, magnesium carbonate and the like.

An inoculum or vaccine composition takes the form of a solution, suspension, tablet, pill, capsule, sustained release formulation or powder, and contains an immunogenic effective amount of HBc chimer, preferably as particles, as active ingredient. In a typical composition, an immunogenic effective amount of preferred HBc chimer particles is about 1 μg to about 1 mg of active ingredient per dose, and more preferably about 5 μg to about 50 μg per dose, as noted before.

A vaccine or inoculum is typically formulated for parenteral administration. Exemplary immunizations are carried out sub-cutaneously (SC) intra-muscularly (IM), intravenusly (IV), intraperitoneally (IP) or intra-dermally (ID).

The HBc chimer particles and HBc chimer particle conjugates can be formulated into the vaccine as neutral or salt forms. Pharmaceutically acceptable salts, include the acid addition salts (formed with the free amino groups of the protein or hapten) and are formed with inorganic acids such as, for example, hydrochloric or phosphoric acids, or such organic acids as acetic, oxalic, tartaric, mandelic, and the like. Salts formed with the free carboxyl groups can also be derived form inorganic bases such as, for example, sodium, potassium, ammonium, calcium, or ferric hydroxides, and such organic bases as isopropylamine, trimethylamine, 2-ethylamino ethanol, histidine, procaine, and the like.

The inocula or vaccines are administered in a manner compatible with the dosage formulation, and in such amount as are therapeutically effective and immunogenic (an antibody-inducing amount or protective amount, as is desired). The quantity to be administered depends on the subject to be treated, capacity of the subject's immune system to synthesize antibodies, and degree of protection desired. Precise amounts of active ingredient required to be administered depend on the judgment of the practitioner and are peculiar to each individual. However, suitable dosage ranges are of the order of several hundred micrograms active ingredient per individual. Suitable regimes for initial administration and booster shots are also variable, but are typified by an initial administration followed in intervals (weeks or months) by a subsequent injection or other administration.

Once immunized, the host animal is maintained for a period of time sufficient for the recombinant HBc chimer immunogen to induce the production of a sufficient titer of antibodies that bind to the M2 protein. The maintenance time for the production of anti-M2 antibodies typically lasts for a period of about three to about twelve weeks, and can include a booster, second immunizing administration of the vaccine. A third immunization is also contemplated, if desired, at a time 24 weeks to five years after the first immunization. It is particularly contemplated that once a protective level titer of antibodies is attained, the vaccinated host animal is preferably maintained at or near that antibody titer by periodic booster immunizations administered at intervals of about 1 to about 5 years.

The production of antibodies is readily ascertained by obtaining a plasma or serum sample from the immunized host and assaying the antibodies therein for their ability to bind to a synthetic M2 polypeptide antigen in an ELISA assay as described hereinafter or by another immunoassay such as a Western blot as is well known in the art.

It is noted that the before-described antibodies so induced can be isolated from the blood of the host using well-known techniques, and then reconstituted into a second vaccine for passive immunization as is also well known. Similar techniques are used for gamma-globulin immunizations of humans. For example, antiserum from one or a number of immunized hosts can be precipitated in aqueous ammonium sulfate (typically at 40-50 percent of saturation), and the precipitated antibodies purified chromatographically as by use of affinity chromatography in which an M2 polypeptide is utilized as the antigen immobilized on the chromatographic column.

Inocula are preparations that are substantially identical to vaccines, but are used in a host animal in which antibodies to influenza are desired to be induced, but in which protection from influenza is not desired.

Without further elaboration, it is believed that one skilled in the art can, using the preceding description and the detailed examples below, utilize the present invention to its fullest extent. The following preferred specific embodiments are, therefore, to be construed as merely illustrative, and not limiting of the remainder of the disclosure in any way whatsoever.

### EXAMPLE 1

### B Cell Epitope-Containing Chimer Preparation

A. Preparation of plasmid vector pKK223-3N, a Modified Form of pKK223-3

Plasmid vector pKK223-3 (Pharmacia) was modified by the establishment of a unique NcoI restriction site to enable insertion of HBc genes as NcoI-HindIII restriction fragments and subsequent expression in E. coli host cells. To modify the pKK223-3 plasmid vector, a new SphI-HindIII fragment was prepared using the PCR primers pKK223-3/433-452-F and pKK223-NcoI-mod-R, and pKK223-3 as the template.

This PCR fragment was cut with the restriction enzymes SphI and HindIII to provide a 467 bp fragment that was then ligated with a 4106 bp fragment of the pKK223-3 vector, replacing the original 480 bp SphI-HindIII fragment. The resultant plasmid (pKK223-3N; 4573 bp) is therefore 13 bp shorter than the parent plasmid and contains modified nucleotide sequence upstream of the introduced NcoI site (see FIG. 2, in which the dashes indicate the absent bases). Restriction sites in plasmid pKK223-3N are indicated in FIG. 2 and nucleotide changes made to the pKK223-3 parent plasmid are indicated by an underline as shown below.

| | | |
|---|---|---|
| pKK223-3/433-452-F | GGTGCATGCAAGGAGATG | SEQ ID NO:26 |
| pKK223-NcoI-mod-R | GCGAAGCTTCGGATCccatggTTTTTTCCTCCTTATGTGAAATTGTTATCCG- | SEQ ID NO:27 |
| CTC | | |

B. Preparation of V1, V2 and V8 Cloning Vectors

Modified HBc149 (V1 and V2) or HBc183 (V8) genes, able to accept the directional insertion of synthetic dsDNA fragments into the immunodominant loop region, were constructed using PCR. (The plasmid accepting inserts between amino acids E77 and D78 and truncated to V149 was named V1, the plasmid accepting inserts between D78 and P79 and truncated to V149 was named V2, and the plasmid accepting inserts between D78 and P79 and terminating at C183, was called V8). The HBc149 and HBc183 genes were amplified in two halves using two PCR primer pairs, one of which amplifies the amino terminus, the other amplifies the carboxyl terminus. For V1, the products of the PCR reactions (N- and C-terminus) are both 246 bp fragments; for V2, the products are a 249 bp (N-terminus) and a 243 bp fragment (C-terminus); for V8, the products are a 249 bp (N-terminus) and a bp fragment (C-terminus).

The N-terminal fragments prepared were digested with NcoI and EcoRI, and the C-terminal fragments were digested with EcoRI and HindIII. The V1, V2 and V8 fragment pairs were then ligated together at the common EcoRI overhangs. The resultant NcoI-HindIII fragments were then ligated into the pKK223-3N vector, which had been prepared by digestion with NcoI and HindIII.

To insert B cell epitopes into the V1, V2 and V8 plasmids, the appropriate plasmid was digested with EcoRI and SacI restriction enzymes. Synthetic dsDNA fragments containing 5′ EcoRI and 3′ SacI overhangs were then inserted. In all cases, V1, V2, and V8, glycine-isoleucine (EcoRI) and glutamic acid-leucine (SacI) amino acid pairs, flank the inserted B cell epitopes. The inserted restriction sites are underlined in the primers below.

V1

| | |
|---|---|
| V1 | |
| HBc149/NcoI-F | |
| 5′-GGGCCATGGACATCGACCCTTA | SEQ ID NO:28 |
| HBc-E77/EcoRI-R | |
| 5′-GCGGAATTCCTTCCAAATTAACACCCACC | SEQ ID NO:29 |
| HBc-D78/EcORI-SacI-F | |
| 5′-CGCGAATTCAAAAAGAGCTCGATCCAGCGTCTAGAGAC | SEQ ID NO:30 |
| HBc149/HindIII-R | |
| 5′-CGCAAGCTTAAACAACAGTAGTCTCCGGAAG | SEQ ID NO:31 |
| V2 | |
| HBc149/NcoI -F | |
| 5′-TTGGGCCATGGACATCGACCCTTA | SEQ ID NO:32 |
| HBc-D78/EcoRI -R | |
| 5′-GCGGAATTCCATCTTCCAAATTAACACCCAC | SEQ ID NO:33 |
| HBc-P79/EcoRI -SacI-F | |
| 5′-CGCGAATTCAAAAAGAGCTCCCAGCGTCTAGAGACCTAG | SEQ ID NO:34 |
| HBc149/HindIII-R | |
| 5′-CGCAAGCTTAAACAACAGTAGTCTCCGGAAG | SEQ ID NO:31 |
| V8 | |
| HBc149/NcoI-F | |
| 5′-GGGCCATGGACATCGACCCTTA | SEQ ID NO:28 |
| HBc-D78/EcoRI-R | |
| 5′-GCGGAATTCCATCTTCCAAATTAACACCCAC | SEQ ID NO:29 |
| HBc-P79/EcoRI-SacI-F | |
| 5′- CGCGAATTCAAAAAGAGCTCCCAGCGTCTAGAGACCTAG | SEQ ID NO:35 |
| HBc183/HindIII-R | |
| 5′-GGAAAGCTTACTAACATTGAGATTCCCG | SEQ ID NO:36 |
| V34/V55 | |
| pKK-BamiHI-F | |
| 5′-GCGGGATCCGGAGCTTATCGA | SEQ ID NO: 37 |
| HBc-NcoI/EcoRI/SacI-R | |
| 5′-GCGGAGCTCTTTTTGAATTCCCATGGTTTTTTCCTCCTTAT | SEQ ID NO:38 |
| PreC-SacI-HBc-F | |
| 5′-GCGGAGCTCCTTGGGTGGCTTTGGGGCATTGACATCGACCCTTATAAAG | SEQ ID NO:39 |
| V34 | |
| HBc149/HindIII-R | |
| 5′-CGCAAGCTTAAACAACAGTAGTCTCCGGAAG | SEQ ID NO:31 |
| V55 | |
| HBc149+C/HindIII-R | |
| 5′- CGCAAGCTTACTAGCAAACAACAGTAGTCTCCGGAAG | SEQ ID NO:40 |

C. Preparation of V34 and V55 Cloning Vectors

Modified HBc149 genes, able to accept the directional insertion of synthetic dsDNA fragments into the N-terminal region, 5′ to the pre-core sequence LGWLWG, were constructed using PCR. (The plasmid that encoded an HBc sequence terminating at V149 was named V34, whereas the plasmid that encoded an HBc sequence harboring an additional cysteine, C-terminal to V149, was named V55.) The HBc149 gene was amplified in two halves using two PCR primer pairs, one of which amplifies the amino terminus (for which V1 was used as a template), the other amplifies the carboxyl terminus. For V34, the products of the PCR reactions were a 293 bp (N-terminus) fragment and a 484 bp (C-terminus) fragment; for V55, the same N-terminal fragment was used and a 490 bp C-terminal fragment was prepared.

The N-terminal fragment prepared by PCR was digested with NcoI and SacI, and the C-terminal fragments were digested with SacI and HindIII. The V34 and V55 fragment pairs were then ligated together at the common SacI overhangs. The resultant NcoI-HindIII fragments were then ligated into the pKK223-3N vector, which had been prepared by digestion with NcoI and HindIII.

B cell epitope insertion was accomplished by a procedure identical to that outlined above for the V1 cloning vector. Restriction sites are underlined in the oligonucleotides primers below.

| | |
|---|---|
| V34/V55 | |
| pKK-BamHI-F | |
| 5′-GCGGGATCCGGAGCTTATCGA | SEQ ID NO:37 |
| HBc-NcoI/EcoRI/SacI-R | |
| 5′-GCGGAGCTCCTTTTTGAATTCCCATGGTTTTTTCCTCCTTAT | SEQ ID NO:38 |
| PreC-SacI-HBc-F | |
| 5′-GCGGAGCTCCTTGGGTGGCTTTGGGGCATTGACATCGACCCTTATAAAG | SEQ ID NO:39 |
| V34 | |
| HBc149/HindIII-R | |
| 5′-CGCAAGCTTAAACAACAGTAGTCTCCGGAAG | SEQ ID NO:31 |
| V55 | |
| HBc149 + C/HindIII-R | |
| 5′-CGCAAGCTTACTAGCAAACAACAGTAGTCTCCGGAAG | SEQ ID NO:40 |

D. Preparation of V47, V48 and V54 Cloning Vectors

Modified HBc149 and HBc183 genes, able to accept the directional insertion of synthetic dsDNA fragments into the N-terminal region between amino acid residues I3 and D4 were constructed using PCR. (The plasmid encoding an HBc chimer terminating at V149 was named V47, the plasmid encoding an HBc chimer harboring an additional cysteine, C-terminal to V149, was named V54, and the plasmid encoding an HBc chimer terminating at C183 was named V48). For V47, V48 and V54, a PCR primer pairs was used to amplify the amino terminus, from the template V1, including sequence preceeding the HBc gene. The resultant PCR fragment of 190 bp for the C-terminus of V47, the HBc gene was amplified using a PCR primer pair resulting in a 469 bp fragment; for V54, the C-terminal fragment is 475 bp. For the C-terminus of V48, the HBc183 gene was amplified using a PCR primer pair, resulting in a 574 bp fragment.

The cloning procedure used from this point was identical to that outlined for the cloning vector V1.

To insert epitopes into the V47, V48 and V54 plasmids, the plasmids were first digested with NcoI and SacI restriction enzymes. Synthetic dsDNA fragments containing 5′ AflIII and 3′ SacI overhangs were then inserted (note, restriction enzymes AflIII and NcoI leave compatible overhangs). In all cases, V47, V48, and V54, HBc residues D2 and I3 were deleted so that the sequence of the epitope directly follows residue M1; the glutamic acid-leucine (EL) amino acid pairs, coded for by the SacI restriction site, follows the inserted epitope. The inserted restriction sites are underlined in the oligonucleotide primers below.

| | |
|---|---|
| V47/V48/V54 | |
| pKK(167-150)-F | |
| 5′-GCATAATTCGTGTCGCTC | SEQ ID NO:41 |
| HBc-I3/EcoRI-R | |
| 5′-GCGGAATTCCGATGTCCATGGTTTTTTCCT | SEQ ID NO:42 |
| HBc-EcoRI/SacI/D4-F | |
| 5′- GCGGAATTCAAAAAGAGCTCGACCCTTATAAAGAATTTGGA | SEQ ID NO:43 |
| V47 | |
| HBc149/HindIII-R | |
| 5′-CGCAAGCTTAAACAACAGTAGTCTCCGGAAG | SEQ ID NO:31 |
| V54 | |
| HBc149+C/HindIII-R | |
| 5′-CGCAAGCTTACTAGCAAACAACAGTAGTCTCCGGAAG | SEQ ID NO:40 |
| V48 | |
| HBc183/HindIII-R | |
| 5′-GGAAAGCTTACTAACATTGAGATTCCCG | SEQ ID NO:36 |

### EXAMPLE 2

### Preparation of Chimers Containing Influenza A M2 Polypeptide Sequences

A. Insertion of Influenza A M2 N-terminal Domain into V34, V47, V48, V54, and V55 Cloning Vectors

| | |
|---|---|
| V47 | |
| M2 (1-24/C17S) | |
| M S L L T E V E T P I R N E W G S R SEQ ID NO:44 | |
| CATGTCTCTGCTGACCGAAGTTGAAACCCCTATCAGAAACGAATGGGGGTCTAGA | SEQ ID NO:45 |
| AGAGACGACTGGCTTCAACTTTGGGGATAGTCTTTGCTTACCCCCAGATCT | SEQ ID NO:46 |
| C N D S S D E L | |
| TGTAACGATTCAAGTGATGAGCT | |
| ACATTGCTAAGTTCACTAC | |
| M2 (1-24/C19S) | |
| M S L L T E V E T P I R N E W G C R | SEQ ID NO:47 |
| CATGTCTCTGCTGACCGAAGTTGAAACCCCTATCAGAAACGAATGGGGGTGCAGA | SEQ ID NO:48 |
| AGAGACGACTGGCTTCAACTTTGGGGATAGTCTTTGCTTACCCCCACGTCT | SEQ ID NO:49 |
| S N D S S D E L | |
| TCGAACGATTCAAGTGATGAGCT | |
| AGCTTGCTAAGTTCACTAC | |

B. Insertion of a mutated Influenza A M2 (M2(2-24/C17A,C19A)into V2, V8 and V16 Expression Vectors

| | |
|---|---|
| V2, V8, V16 | |
| M2 (2-24/C17A,C19A) | |
| I S L L T E V E T P I R N E W G A R | SEQ ID NO:50 |
| ATTTCTCTGTTAACCGAAGTGGAGACGCCGATTCGTAACGAATGGGGTGCGCGC | SEQ ID NO:51 |
| AGAGACAATTGGCTTCACCTCTGCGGCTAAGCATTGCTTACCCCACGCGCG | SEQ ID NO:52 |
| A N D S S D E L | |
| GCCAATGATAGCTCTGACGAGCT | |
| CGGTTACTATCGAGACTGC | |

C. Insertion of Influenza A M2 N-terminal Domain into Vectors V34, V47, V54, and V55

For V34 and V55 constructs, synthetic dsDNA fragments coding for the M2 epitope (residues 1-24 of the influenza A M2 protein; SEQ ID NO: 9) were inserted into EcoRI/SacI restriction sites, whereas for V47 and V54 constructs, the same were inserted into NcoI/SacI restriction sites. Synthetic dsDNA fragments were prepared by mixing complementary single stranded DNA oligonucleotides at equimolar concentrations, heating to 95° C. for 5 minutes, and then cooling to room temperature at a rate of −1° C. per minute. This annealing reaction was performed in TE buffer. The double-stranded DNAs are shown below with the encoded epitope sequence shown above.

| | |
|---|---|
| V34/V55 | |
| M2 (2-24) | |
| I S L L T E V E T P I R N E W G C R | SEQ ID NO:53 |
| AATTAGCCTGTTAACCGAAGTGGAGACGCCGATCCGTAACGAATGGGGCTGCCG | SEQ ID NO:54 |
| TCGGACAATTGGCTTCACCTCTGCGGCTAGGCATTGCTTACCCCGACGGC | SEQ ID NO:55 |
| C N D S S D E L | |
| CTGTAATGATTCTTCCGACGAGCT | |
| GACATTACTAAGAAGGCTGC | |
| V47/V54 | |
| M2 (1-24) | |
| M S L L T E V E T P I R N E W G C R | SEQ ID NO:56 |
| CATGTCTCTGCTGACCGAAGTTGAAACCCCTATCAGAAACGAATGGGGGTGCAGA | SEQ ID NO:57 |
| AGAGACGACTGGCTTCAACTTTGGGGATAGTCTTTGCTTACCCCCACGTCT | SEQ ID NO:58 |
| C N D S S D E L | |
| TGTAACGATTCAAGTGATGAGCT | |
| ACATTGCTAAGTTCACTAC | |
| M2 (1-24/C17S,C19S) | |
| M S L L T E V E T P I R N E W G S R | SEQ ID NO:59 |
| CATGTCTCTGCTGACCGAAGTTGAAACCCCTATCAGAAACGAATGGGGGTCTAGA | SEQ ID NO:60 |
| AGAGACGACTGGCTTCAACTTTGGGGATAGTCTTTGCTTACCCCCAGATCT | SEQ ID NO:61 |
| S N D S S D E L | |
| TCGAACGATTCAAGTGATGAGCT | |
| AGCTTGCTAAGTTCACTAC | |

D. Construction of Truncated Version of Native M2-HBc

The original M2-HBc construct [Neirynck et al., (October 1999) Nature Med., 5(10):1157-1163: WO 99/07839] that contained the 183-residue, full length HBc sequence was truncated to V149, and the entire gene was moved into the pKK223-3 expression vector. To achieve this, the plasmid 3453, which was provided by the University of Gent, was used as a template for a PCR reaction that yielded a product of 523 bp. This product was digested with restriction enzymes AflIII and HindIII, and then ligated into the pKK223-3N vector, which had been prepared by digestion with NcoI and HindIII.

| | |
|---|---|
| AflIII-M2-F | |
| 5′-CGCGACATGTCTCTGCTGACCG | SEQ ID NO:62 |
| HBc-HindIII-R | |
| 5′-CGCAAGCTTAAACAACAGTAGTCTCCGGAAG | SEQ ID NO:31 |

### EXAMPLE 3

### Vectors to Express Chimers Displaying Both N-terminal- and Loop-Inserted Influenza M2 Sequences, Stabilized at the N-Terminus, or both N- and C-Termini

A. Vector to Express Hybrid Particles with both an N-terminal Fusion and a Loop Insertion, Stabilized at the N-terminus

To construct a vector containing two copies of the influenza M2 sequence, a copy of native M2 (1-24) at the N-terminus and a mutated version of M2 [M2(2-24/C17A,C19A)] in the immunodominant loop, the vector V47.M2(1-24)/V2.M2(2-24/C17A,C19A) was constructed. This vector was constructed by digesting vector V47.M2(1-24) with the restriction enzymes BglII and HindIII, isolating the digested vector, and inserting a 449 bp BglII-HindIII fragment from V2.M2(2-24/C17A,C19A).

B. Vector to Express Hybrid Particles with both an N-terminal Fusion and a Loop Insertion, stabilized at both the N- and C-termini

To construct a vector containing two copies of the influenza M2 sequence, a copy of native M2 (1-24) at the N-terminus, a mutated version of M2 [M2(2-24/C17A,C19A)] in the immunodominant loop, and a C-terminal cysteine, the vector V47.M2(1-24)/V16.M2(2-24/C17A,C19A) was constructed. This vector was constructed by digesting vector V47.M2(1-24) with the restriction enzymes BglII and HindIII, isolating the digested vector, and inserting a 452 bp BglII-HindIII fragment from V16.M2(2-24/C17A,C19A).

### EXAMPLE 4

### Assay Procedures

A. Antigenicity

1. Particle ELISA

Purified particles were diluted to a concentration of 10 μg/mL in coating buffer (50 mM sodium bicarbonate, pH 9.6) and coated onto the wells of ELISA strips (50 μL/well). The ELISA strips were incubated at room temperature overnight (about 18 hours). Next morning, the wells were washed with ELISA wash buffer [phosphate buffered saline (PBS), pH 7.4, 0.05% Tween®-20] and blocked with 3% BSA in PBS for 1 hour (75 μL/well). ELISA strips were stored, dry, at −20° C. until needed.

To determine the antigenicity of particles, antisera were diluted using 1% BSA in PBS and 50 μL/well added to antigen-coated ELISA wells. Sera were incubated for 1 hour, washed with ELISA wash buffer (above) and probed using an anti-mouse (IgG)-HRP (The Binding Site, San Diego, Calif.; HRP=horseradish peroxidase) conjugate (50 μL/well) or other appropriate antibody for 30 minutes. After washing with ELISA wash buffer the reaction was visualized by the addition of TM blue substrate (50 μL/well). After 10 minutes, the reaction was stopped by the addition of 1N H₂SO₄ (100 μL/well) and read on an ELISA plate reader set at 450 nm.

2. Synthetic Peptide ELISA

A 24 amino acid residue synthetic peptide M2 is diluted to a concentration of 2 μg/mL in coating buffer (50 mM sodium bicarbonate, pH 9.6) and coated onto the wells of ELISA strips (50 μL/well). Peptides are dried onto the wells by incubating overnight (about 18 hours), in a hood with the exhaust on. Next morning, the wells are washed with ELISA wash buffer (phosphate buffered saline, pH 7.4, 0.05% Tween®-20) and blocked with 3% BSA in PBS (75 μL/well) for 1 hour. ELISA strips are stored, dry, at −20° C. until needed.

To determine antibody antigenicity of particles, antisera (monoclonal or polyclonal) are diluted using 1% BSA in PBS, and 50 μL/well added to antigen-coated ELISA wells. Sera are incubated for 1 hour, washed with ELISA wash buffer, and probed using an anti-mouse(IgG)-HRP conjugate or other antibody (as above at 50 μL/well) for 30 minutes, washed again with ELISA wash buffer, and then visualized by the addition of TM blue substrate (50 μL/well). After 10 minutes, the reaction is stopped by the addition of 1N H₂SO₄ (100 μL/well) and read on an ELISA plate reader set at 450 nm.

B. Immunogenicity of Particles

To assay the immunogenicity of particles, mice are immunized, IP, with 20 μg of particles in Freund's complete adjuvant, and then boosted at 4 weeks with 10 μg in Freund's incomplete adjuvant. Mice were bled at 2, 4, 6, and 8 weeks.

### EXAMPLE 5

### Determination of 280:260 Absorbance Ratios

To determine the 280:260 absorbance ratio of purified particles, the particles were diluted to a concentration of approximately 0.2 mg/mL in 20 mM sodium phosphate buffer, pH 6.8, and absorbance values determined at wavelengths of 260 and 280 nm. The absorbance measured at 280 nm was divided by the value at 260 nm to determine the 280:260 ratio. The ratios were obtained for several samples, including native particles (HBc183), HBc particles truncated after residue position 149 (HBc149), and several HBc chimers that are identified elsewhere herein, are shown below in Table 1. Full length particles ICC-1559 are a preparation of the particles first reported in Neirynck et al., (October 1999) Nature Med., 5(10):1157-1163, whereas full length particles ICC-1607 are similar particles in which the M2 polypeptide cysteines at polypeptide positions 17 and 19, (X₁₇ and X₁₉ of SEQ ID NO: 9) were mutated to serine residues.

TABLE 1

| | Full Length, (F) | |
|---|---|---|
| | or | 280/260 |
| Particle | C-Terminal | Absorbance |
| Number | Truncated, (T) | Ratio |
| HBc183 | F | 0.84 |
| HBc149 | T | 1.59 |
| 1438 | T | 1.57 |
| 1473 | T | 1.64 |
| 1475 | T | 1.04 |
| 1492 | T | 1.33 |
| 1559 | F | 0.68 |
| 1560 | T | 1.36 |
| 1590 | T | 1.51 |
| 1603 | T | 1.68 |
| 1604 | T | 1.40 |
| 1605 | T | 1.26 |
| 1607 | F | 0.73 |

### EXAMPLE 6

### Thermal Stability Protocol

Purified particles were diluted to a concentration of 1 mg/mL using 50 mM NaPO₄, pH 6.8 and sodium azide was added to a final concentration of 0.02% to prevent bacterial growth. Samples were mixed with SDS-PAGE sample buffer (reducing) and run on 15% SDS-PAGE gels. Gels were stained using Coomassie Blue, and then analyzed.

### EXAMPLE 7

### Analytical Gel Filtration Analysis of Hybrid Particles

Analytical gel filtration analysis of purified hybrid HBc particles was performed using a 25 mL Superose ® 6 HR 10/30 chromatographic column (Amersham Pharmacia # 17-0537-01) and a BioCAD™ SPRINT Perfusion Chromatography System. The UV detector was set to monitor a wavelength of 280 nm. The column was equilibrated with 3 column volumes (CV; about 75 mL) of buffer (50 mM NaPO₄, pH 6.8) at a flow rate of 0.75 mL/minute.

The particles to be analyzed were diluted to a concentration of 1 mg/mL using 50 mM NaPO₄, pH 6.8. 200 Microliters (μL) of the sample were then loaded onto a 200 μL loop and injected onto the column. The sample was eluted from the column with 50 mM NaPO₄, pH 6.8 at a flow rate of 0.75 mL/minute.

Particles containing N-terminal cysteine residues or similar particles free of such cysteines were analyzed using the above procedure. Integration of the 280 nm trace was carried out using BioCAD™ software (PerSeptive™) to provide the results in

### EXAMPLE 8

### Influenza M2 Constructs

Recently, Neirynck et al., (October 1999) Nature Med., 5(10):1157-1163 and WO 99/07839 reported the fusion of the 24 amino acid extracellular domain of M2 to the N-terminus of full-length HBc particles (HBc183), lacking amino acid residues 1-4. A schematic representation of that construct referred to herein as IM2HBc is shown below in which the 24-mer is linked to the N-terminus of HBc.

| | |
|---|---|
| IM2HBc | |
| MSLLTEVETPIRNEWGCRCNDSSD-HBc (5-183) | SEQ ID NO:63 |

In one illustrative preparation, the M2 epitope was inserted into the immunodominant loop of hepatitis B core and particles referred to as ICC-1475 were successfully expressed and purified using techniques discussed previously for such insertions and purifications. A mutated version of the M2 epitope, in which two cysteine residues at M2 native positions 17 and 19 were substituted by alanine residues, was also expressed in the immunodominant loop (ICC-1473 particles) and the resulting particles purified. These two particles are illustrated schematically below.

| | |
|---|---|
| ICC-1475 | |
| HBc (1-78)-GI-SLLTEVETPIRNEWGCRCNDSSD- | SEQ ID NO: 64 |
| EL-HBc(79-149) | |
| ICC-1473 | |
| HBc(1-78)-GI-SLLTEVETPIRNEWGARANDSSD- | SEQ ID NO: 65 |
| EL-HBc(79-149)-C | |

The ICC-1473 particle construct yielded approximately 7-fold more purified particles when compared with the native sequence (ICC-1475). It remains to be determined if the mutation of the cysteine residues alters protective potential of the particles. However, epitopes delivered on the immunodominant loops of HBc are usually significantly more immunogenic as compared to when they are fused to other regions (including the N-terminus), and resulting particles exhibit reduced anti-HBc immunogenicity.

Particles have also been prepared in which the M2 N-terminal 24-mer epitope was fused to the N-terminus of C-terminal truncated hepatitis B core particles. That construct (ICC-1438) also contained the N-terminal pre-core sequence (SEQ ID NO: 66). A similar construct was prepared that contained a single cysteine residue at the end of the hybrid protein (ICC-1492), in this case immediately after Val-149 of the HBc gene. These constructs are shown schematically below.

| | |
|---|---|
| ICC-1438 | SEQ ID NO:66 |

MGISLLTEVETPIRNEWGCRCNDSSDELLGWLWGI-HBc (2-149)

| | |
|---|---|
| ICC-1492 | SEQ ID NO:67 |

MGISLLTEVETPIRNEWGCRCNDSSDELLGWLWGI-HBc (2-149)-C

It should be noted that to guard against translation initiation from the natural HBc initiator methionine, the codon for that residue was mutated to code for an isoleucine residue. Residues contributed by EcoRI (GI) and SacI (EL) restriction sites are underlined. The pre-core sequence is recited between the underlined EL residues and “-HBc(2-149)”.

Analysis by SDS-PAGE as discussed elsewhere herein, showed that upon preparation, the ICC-1438 monomer construct was unstable (Lane 2) as compared to the ICC-1492 (Lane 3), with HBc-149 (Lane 1), ICC-1475 (Lane 4) and ICC-1473 (Lane 5) serving as additional molecular weight controls on the SDS-PAGE gel in FIG. 10. The instability of the ICC-1438 monomers was not evident using analytical gel filtration of particles.

Both ICC-1475 (FIG. 10, lane 4) and ICC-1473 (FIG. 10, lane 5) were expected to have slightly lower molecular weights than ICC-1438 and ICC-1492, because the former two contain the M2 epitope inserted directly into the immunodominant loop and therefore lack the pre-core sequence (SEQ ID NO: 66) present in ICC-1438 and ICC-1498. As expected, ICC-1492 was larger than ICC-1475 and ICC-1473; however, ICC-1438, which is identical to ICC-1492 save the C-terminal cysteine residue, is clearly not larger than ICC-1475 and ICC-1473 due to an apparent cleavage.

A construct containing a M2 N-terminal extracellular sequence as discussed above linked to the HBc N-terminus (Domain I) or loop (Domain II) and also containing a M2 protein C-terminal sequence such as that of SEQ ID NO: 11 (see Table A) linked the loop (Domain II) or at the C-terminus (Domain IV) of HBc is also contemplated. Such a contemplated construct also contains at least one stabilizing C-terminal cysteine residue as discussed elsewhere herein.

To modify the amino-terminus of hybrid HBc particles containing immunodominant loop fusions to incorporate a cysteine residue, and minimal M2-derived sequence, a series of synthetic oligonucleotides are synthesized. To make V2.Pf1(N-M2(17-24/C17S), the oligonucleotides M2(17-24/C17S)-NcoI-F and HBc149/HindIII-R are used to amplify the hybrid HBc gene from vector V2.Pf1. The resultant 546 bp fragment is cleaved with NcoI and HindIII and inserted into pKK-223-3N, which has been cleaved with the same two enzymes.

To make V2.Pf1(N-M2(17-24/C19S), the oligonucleotides M2(17-24/C19S)-NcoI-F and HBc149/HindIII-R are used to amplify the hybrid HBc gene in vector V2.Pf1. The resultant 540 bp fragment is cleaved with NcoI and HindIII and inserted into pKK-223-3N, which had been cleaved with the same two enzymes.

| | |
|---|---|
| M2 (17-24/C17S)-NcoI-F | |
| M G S R C N D S S D I D P Y K E F G | SEQ ID NO:68 |
| GGCGCCATGGGGTCTAGATGTAACGATTCAAGTGACATCGACCCTTATAAAGAATTTCG | SEQ ID NO:69 |
| M2 (17-24/C19S)-NcoI-F | |
| M G C N D S S D I D P Y K E F G | SEQ ID NO:70 |
| GCGCCATGGGGTGTAACGATTCAAGTGACATCGACCCTTATAAAGAATTTGG | SEQ ID NO:71 |

### EXAMPLE 9

### HBc Chimer Molecules With and Without Both N- and C-Terminal Cysteine Residues

A series of HBc chimer molecule-containing particles was prepared that contained residues 1-24 of the influenza A, M2 protein peptide-bonded at or near the N-terminus of HBc whose C-terminus was truncated at residue 149. The component chimeric protein molecules contained different N-terminal sequences that included an M2 sequence or variant, and some contained a C-terminal cysteine residue.

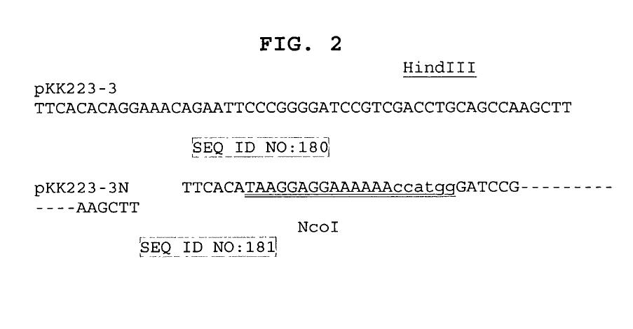

All purified particles listed in Table 2, below, were analyzed by analytical size exclusion chromatography to assess the retention of particulate structure following purification. Particles designated ICC-1603, which contain no N-terminal cysteine residues, displayed evidence of disassembly back to sub-particulate structures (FIG. 3) because the protein eluted in the 1500 second range (particles elute at approximately 1000 seconds).

Similar analysis of particles ICC-1590, which are similar to ICC-1603 ICC-particles except for the mutation of two serine residues to cysteine residues in the N-terminal M2 sequence, revealed that that construct remained particulate following purification, with elution occurring at around 1000 seconds, which is typical for a hybrid particle (FIG. 4). There was no evidence of disassembly for ICC-1590 particles.

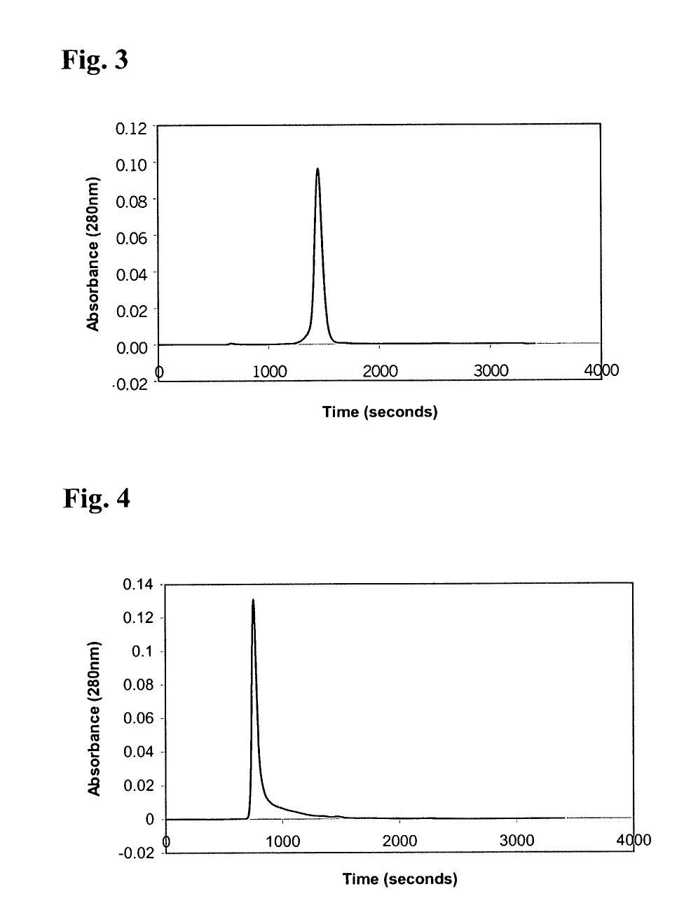

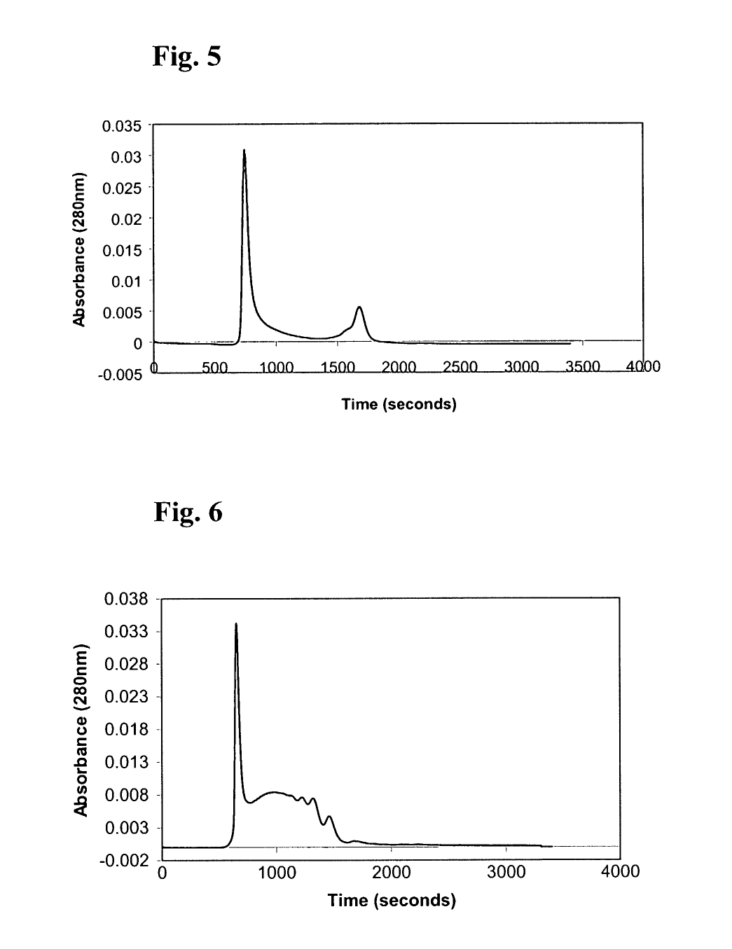

Analysis of ICC-1560 particles, whose chimer protein also has two N-terminal cysteine residues, revealed that it too was particulate following purification, although it did exhibit some degree of disassembly (FIG. 5), suggesting that the stabilization was not quite as robust as it was for ICC-1590 particles. Comparison of the N-terminal configurations of ICC-1590 and ICC-1560 particles (Table 2, hereinafter), shows that the relative position of the two cysteine residues in ICC-1560 particles is shifted by 3 amino acid residues relative to ICC-1590 particles via the deletion of three amino acid residues (DEL), indicating that the cysteine residues may be required to be a minimal distance from the start of the core gene to enable optimal cross-linking.

### EXAMPLE 10

### Particles With an M2 or M2 Variant Sequence and A C-Terminal Cysteine Residue

ICC-1603 particles were shown in FIG. 3 to rapidly disassemble following purification. The HBc chimer molecules that comprise ICC-1605 particles are similar to those of ICC-1603 particles, except that the ICC-1605 component chimer molecules have a single C-terminal stabilizing cysteine. A plasmid was made to direct the expression of ICC-1605 particles to investigate if the addition of a C-terminal cysteine residue to ICC-1603 particles could impart greater stability on the particle. Following purification, ICC-1605 particles were analyzed using analytical size exclusion chromatography (FIG. 6).

The results of this study demonstrated that particle stabilization was more complete than for the ICC-1603 particles, but incomplete compared to ICC-1590 particles, which contains two amino-terminal cysteine residues and no C-terminal stabilizing cysteine. Although a significant amount of ICC-1605 remained particulate, there was evidence of a heterogeneous mixture of sub-particulate structures that eluted over a broad range. These observations suggest that for this hybrid particle (ICC-1603), C-terminal stabilization as found in ICC-1605 particles was less complete than for the N-terminal stabilization found in ICC-1590 particles.

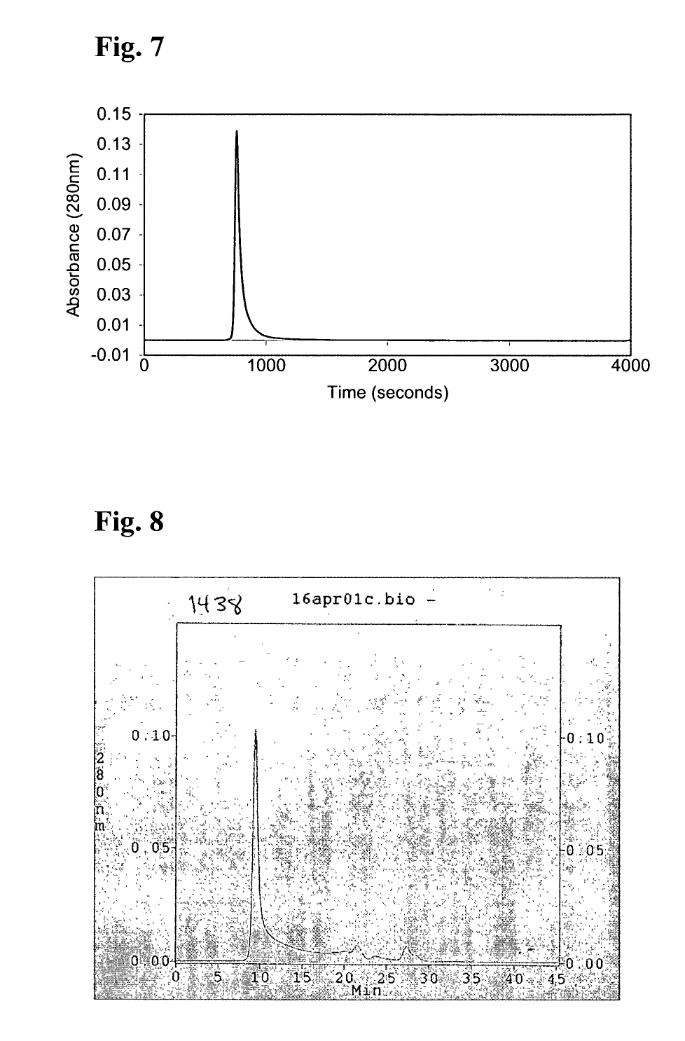

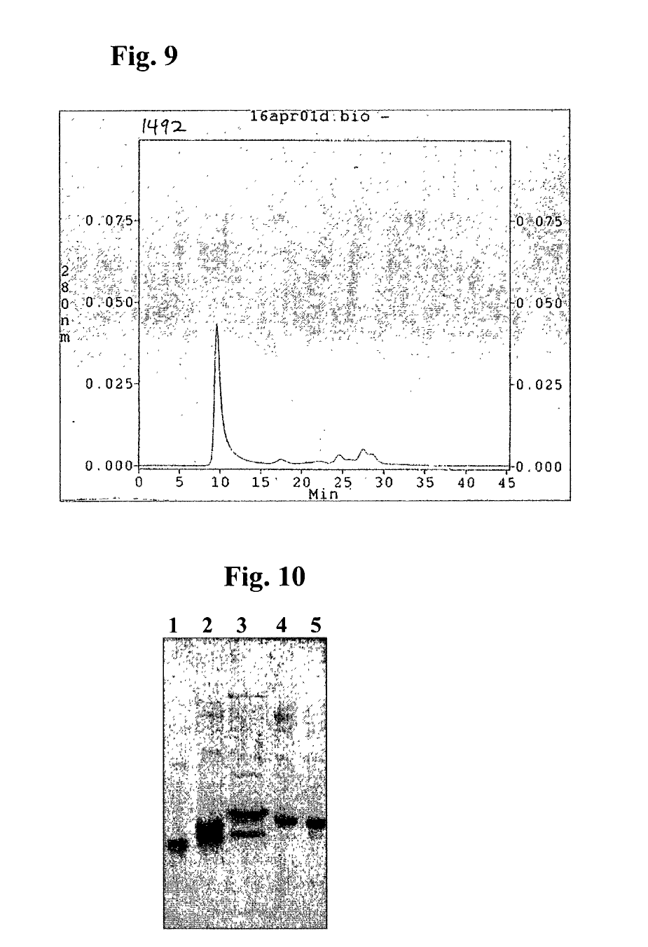

To investigate the compatibility of combined amino and carboxyl-terminal cysteine stabilization of hybrid particles, an expression plasmid was constructed to direct the expression of ICC-1604 particles. The component chimer molecules of ICC-1604 particles contain both the two amino-terminal stabilizing cysteine residues present in a native M2 polypeptide sequence (as in ICC-1590) as well as a C-terminal stabilizing cysteine (as in ICC-1605 particles). Analysis of ICC-1604 particles showed that they retained a homogeneous particulate state following purification (FIG. 7), indicating that the two stabilizing methods are complementary and can be used in concert with each other.

Alternative linker sequences between the N-terminus of HBc and the N-terminal cysteine residues were investigated using particles ICC-1438 and ICC-1492. Both of these particles contain the amino acid sequence ELLGWLWGIDI (SEQ ID NO: 72) between the M2 fusion and amino acid D4 of HBc. Amino acid residues LGWLWGIDI are derived from amino acids −6 of pre-core to amino acid I3 of HBc, with the initiator codon of HBc mutated to an isoleucine to prevent translation initiation from this position, which would compromise the study. The HB pre-core sequence includes a cysteine at position −7.

These particles differed only in the fact that the ICC-1438 component chimer molecule terminated at position 149 of HBc, whereas the ICC-1492 component chimer molecule terminated at 149 of HBc and contained a terminal cysteine at position 150 relative to the HBc of SEQ ID NO: 1. When analyzed by analytical gel filtration, using an alternative but similar method to that discussed before, whereby particles elute at approximately 10 minutes, both constructs were shown to be particulate following purification (ICC-1438 in FIG. 8 and ICC-1492 in FIG. 10). This study demonstrated the compatibility of amino- and carboxyl-terminal cysteine stabilization of truncated particles, and the tolerance of substantial variability in the amino acid sequence and distance between the N-terminal cysteine residues and start of the HBc gene.

TABLE 2

| | | | Residues | | Bound | C-term |
|---|---|---|---|---|---|---|
| | | | Between M2 | C-term | Nucleic | Cysteine |
| Construct Name | N-terminal Fusion | HBc N-term Start | and HBc | End | Acid | Stab |
| ICC-1560 | M2 (1-24) | D4 | None | 149 | No | No |
| ICC-1603 | M2 (1-24) | D4 | EL | 149 | No | No |
| | (2C > 2S) | | | | | |
| ICC-1590 | M2 (1-24) | D4 | EL | 149 | No | No |
| ICC-1604 | M2 (1-24) | D4 | EL | 149 | No | Yes |
| | | | | | | (C150) |
| ICC-1605 | M2 (1-24) | D4 | EL | 149 | No | Yes |
| | (2C > 2S) | | | | | (C150) |
| ICC-1438 | M2 (1-24) | D2 | ELLGWLWGI | 149 | No | No |
| ICC-1492 | M2 (1-24) | D2 | ELLGWLWGI | 149 | No | Yes |
| | | | | | | (C150) |

Table 3, below, shows an alignment that illustrates the configuration of the N-termini of HBeAg, and particles designated ICC-1590, ICC-1560, ICC-1603, ICC-1604 and ICC-1605. Sequences are aligned according to amino acid residue position 4 from the N-terminus of HBc of SEQ ID NO:1 that is shared by all constructs. N-terminal cysteine residues, when present, are underlined.

TABLE 3

| Construct Name | Sequence | SEQ ID NO |
|---|---|---|
| HBeAg | SKLCLGWLWGMDID | 73 |
| ICC-1590/ICC-1604 | MSLLTEVETPIRNEWGCRCNDSSDELD | 74 |
| ICC-1560 | MSLLTEVETPIRNEWGCRCNDSSD | 18 |
| ICC-1603/ICC-1605 | MSLLTEVETPIRNEWGSRSNDSSDELD | 75 |
| ICC-1438/ICC-1492 | MGISLLTEVETPIRNEWGCRCNDSSDELLGWLWGIDID | 76 |

Table 4, below, provides a tabulation of the results in which stability was assessed for particles containing an N-terminal influenza A M2 sequence or variant contemplated herein. As is seen, stable particles have been prepared from HBc chimer molecules that contain an N-terminal cysteine residue at a position of minus 14 (−14) relative to the N-terminus of the HBc sequence of SEQ ID NO: 1 to about the N-terminus itself.

TABLE 4

| | Amino Acids Between | | | |
|---|---|---|---|---|
| | HBc D4 and N-terminal | | C-terminal | Stable |
| Construct | Cysteine Residues | | Cysteine | Particle |
| Name | Cys 1 | Cys 2 | Stabilization | Formed |
| HBeAg | — | 9 | No | No |
| ICC-1603 | — | — | No | No |
| ICC-1605 | — | — | Yes | Yes/No |
| ICC-1590 | 9 | 7 | No | Yes |
| ICC-1604 | 9 | 7 | Yes | Yes |
| ICC-1560 | 6 | 4 | No | Yes |
| ICC-1438 | 18 | 16 | No | Yes |
| ICC-1492 | 18 | 16 | Yes | Yes |

Each of the patents and articles cited herein is incorporated by reference. The use of the article “a” or “an” is intended to include one or more.

The foregoing description and the examples are intended as illustrative and are not to be taken as limiting. Still other variations within the spirit and scope of this invention are possible and will readily present themselves to those skilled in the art.

What is claimed:

1. A recombinant hepatitis B virus core (HBc) protein chimer molecule with a length of about 150 to about 325 amino acid residues that contains four peptide-linked amino acid residue sequence domains from the N-terminus that are denominated Domains I, II, III and IV, and includes at least one peptide-bonded polypeptide of about 6 to about 24 residues of the influenza A M2 polypeptide of SEQ ID NO: 9 wherein
(a) Domain I comprises (i) about 75 to about 110 amino acid residues whose sequence includes at least the sequence of the residues of position 4 through position 75 of HBc, (ii) one to three cysteine residues present at a position in the chimer molecule of about one to about −20 relative to the N-terminus of HBc of SEQ ID NO: 1 [N-terminal cysteine residue(s)], said one or more N-terminal cysteine residues being present within a sequence other than that of the pre-core sequence of HBc, and optionally includes said sequence of about 6 to about 24 residues of the influenza A M2 polypeptide of SEQ ID NO: 9 that, when present, is peptide-bonded to or within about 15 residues of the N-terminus of the HBc sequence,
(b) Domain II comprises about 10 to about 60 amino acid residues peptide-bonded to residue 75 of which (i) zero to all of the sequence of HBc is present from position 76 through 85 and (ii) an optional sequence of 6 to about 48 residues that constitute one or more repeats of the influenza A M2 polypeptide of SEQ ID NO: 9;
(c) Domain III is an HBc sequence from position 86 through position 135 peptide-bonded to residue 85; and
d) Domain IV comprises (i) the residues of positions 136-140 plus up to nine residues of an HBc amino acid residue sequence from position 141 through 149 peptide-bonded to the residue of position 135 of Domain III, (ii) zero to three cysteine residues, and (iii) up to about 100 amino acid residues in a sequence heterologous to HBc from position 164 to the HBc C-terminus;
said chimer molecule (i) containing no more than 10 percent conservatively substituted amino acid residues in the HBc sequence, (ii) self-assembling into particles that are substantially free of binding to nucleic acids on expression in a host cell, and said particles being more stable on formation than are particles formed from an otherwise identical HBc chimer that lacks said N-terminal cysteine residue(s) or in which an N-terminal cysteine residue present in the chimer molecule is replaced by another residue.

2. The recombinant HBc chimer protein molecule according to claim 1 wherein one of said residues X₁₇ and X₁₉ of said M2 polypeptide of SEQ ID NO: 9 is cysteine.

3. The recombinant HBc chimer protein molecule according to claim 1 wherein said M2 polypeptide of SEQ ID NO: 9 includes residues X₂ through X₂₄.

4. The recombinant HBc chimer protein molecule according to claim 1 wherein M2 polypeptide of SEQ ID NO: 9 is present in Domain I.

5. The recombinant HBc chimer protein molecule according to claim 1 wherein M2 polypeptide of SEQ ID NO: 9 is present in Domain II.

6. The recombinant HBc chimer protein molecule according to claim 1 wherein Domain I consists essentially of the HBc sequence from position 2 through position 75.

7. The recombinant HBc chimer protein molecule according to claim 1 wherein Domain IV contains zero cysteine residues.

8. The recombinant HBc chimer protein molecule according to claim 1 wherein Domain IV is free of said sequence heterologous to HBc at position 164 to the C-terminus.

9. A recombinant hepatitis B virus core (HBc) protein chimer molecule with a sequence of about 155 to about 225 amino acid residues that contains four peptide-linked domains from the N-terminus that are denominated Domains I, II, III and IV, and includes at least one peptide-bonded polypeptide of about 6 to about 24 residues of the influenza A M2 polypeptide of SEQ ID NO: 9 wherein
(a) Domain I comprises (i) about 95 to about 100 amino acid residues whose sequence includes at least the sequence of the residues of position 4 through position 75 of HBc, (ii) one to three cysteine residues present at a position in the chimer molecule of about one to about −14 relative to the N-terminus of HBc of SEQ ID NO: 1 [N-terminal cysteine residue(s)], said one or more N-terminal cysteine residues being present within a sequence other than that of the pre-core sequence of HBc, and optionally includes said sequence of about 6 to about 24 residues of the influenza A M2 polypeptide of SEQ ID NO: 9 that, when present, is peptide-bonded to or within about 15 residues of the N-terminus of the HBc sequence,
(b) Domain II comprises about 10 to about 60 amino acid residues peptide-bonded to residue 75 of which (i) zero to all of the sequence of HBc is present from position 76 through 85 and (ii) an optional sequence of 6 to about 48 residues that constitute one or more repeats of the influenza A M2 polypeptide of SEQ ID NO: 9;
(c) Domain III consists essentially of the HBc sequence from position 86 through position 135; and
d) Domain IV comprises (i) the residues of positions 136-140 plus up to nine residues of an HBc amino acid residue sequence from position 141 through 149 peptide-bonded to the residue of position 135 of Domain III, (ii) zero or one cysteine residue, and (iii) up to about 50 amino acid residues in a sequence heterologous to HBc from position 164 to the HBc C-terminus;
said chimer molecule (i) containing no more than 5 percent conservatively substituted amino acid residues in the HBc sequence, (ii) self-assembling into particles that are substantially free of binding to nucleic acids on expression in a host cell, and said particles being more stable on formation than are particles formed from an otherwise identical HBc chimer that lacks said N-terminal cysteine residue(s) or in which an N-terminal cysteine residue present in the chimer molecule is replaced by another residue.

10. The recombinant HBc chimer protein molecule according to claim 9 wherein Domain IV includes a sequence of about nine amino acid residues of the HBc sequence from residue position 141 through about position 149 peptide-bonded to residue 140.

11. The recombinant HBc chimer protein molecule according to claim 9 wherein Domain I includes one N-terminal cysteine residue.

12. The recombinant HBc chimer protein molecule according to claim 9 wherein Domain IV includes one C-terminal cysteine residue.

13. The recombinant HBc chimer protein molecule according to claim 9 wherein Domain I includes one N-terminal cysteine residue and Domain IV includes one C-terminal cysteine residue.

14. The recombinant HBc chimer protein molecule according to claim 9 wherein one of said residues X₁₇ and X₁₉ of said M2 polypeptide of SEQ ID NO: 9 is cysteine.

15. The recombinant HBc chimer protein molecule according to claim 9 wherein said M2 polypeptide of SEQ ID NO: 9 includes residues X₂ through X₂₄.

16. The recombinant HBc chimer protein molecule according to claim 9 wherein Domain II comprises the amino acid residues of the sequence of HBc from position 76 through 85.

17. The recombinant HBc chimer protein molecule according to claim 9 wherein Domain II comprises the amino acid residues of the sequence of HBc from position 76 through 85 that further includes 6 to about 23 residues of the influenza A M2 polypeptide of SEQ ID NO: 9.

18. The recombinant HBc chimer protein molecule according to claim 9 wherein Domain I includes 6 to about 23 residues of the influenza A M2 polypeptide of SEQ ID NO: 9.

19. The recombinant HBc chimer protein molecule according to claim 9 that has a length of about 155 to about 170 residues.

20. Particles comprised of recombinant hepatitis B virus core (HBc) protein chimer molecules according to claim 1.

21. Particles comprised of recombinant hepatitis B virus core (HBc) protein chimer molecules according to claim 20 that include the C-terminal 23 residues of the influenza A M2 polypeptide of SEQ ID NO: 9.

22. Particles comprised of recombinant hepatitis B virus core (HBc) protein chimer molecules according to claim 9.

23. Particles comprised of recombinant hepatitis B virus core (HBc) protein chimer molecules according to claim 22 that include the C-terminal 23 residues of the influenza A M2 polypeptide of SEQ ID NO: 9..

24. A vaccine or inoculum comprising an immunogenic effective amount immunogenic particles according to claim 1 dissolved or dispersed in a pharmaceutically acceptable diluent.

25. A vaccine or inoculum comprising an immunogenic effective amount immunogenic particles according to claim 9 dissolved or dispersed in a pharmaceutically acceptable diluent.

26. A nucleic acid that encodes a recombinant HBc protein molecule according to claim 1, or a variant, analog or complement thereof.

27. A nucleic acid that encodes a recombinant HBc protein molecule according to claim 9, or a variant, analog or complement thereof.

28. A recombinant nucleic acid molecule that comprises a vector operatively linked to a nucleic acid segment defining a gene that encodes a recombinant HBc protein molecule according to claim 1, or a varient, analog or complement thereof, and a promoter suitable for driving the expression of the gene in a compatible host organism.

29. A recombinant nucleic acid molecule that comprises a vector operatively linked to a nucleic acid segment defining a gene that encodes a recombinant HBc protein molecule according to claim 9, or a varient, analog or complement thereof, and a promoter suitable for driving the expression of the gene in a compatible host organism.

30. A host cell transformed with a recombinant nucleic acid molecule according to claim 28.

31. The transformed host cell according to claim 30 wherein said host cell is selected from the group consisting of E. coli, S. typhi, S. typhimurium and a S. typhimurium-E. coli hybrid.

32. A host cell transformed with a recombinant nucleic acid molecule according to claim 29.

33. The transformed host cell according to claim 32 wherein said host cell is selected from the group consisting of E. coli, S. typhi, S. typhimurium and a S. typhimurium-E. coli hybrid.
